(19) United States
(12) Patent Application Publication
(10) Pub. No.: US 2025/0262271 A1
(43) Pub. Date: Aug. 21, 2025

(54) AIT1 PROTEIN AND METHODS OF CONTROLLING EUKARYOTIC METABOLISM

(71) Applicant: ARIZONA BOARD OF REGENTS ON BEHALF OF THE UNIVERSITY OF ARIZONA, Tucson, AZ (US)

(72) Inventors: Andrew P. CAPALDI, Tuscon, AZ (US); Ryan L. WALLACE, Tuscon, AZ (US); ERIC LU, Tuscon, AZ (US)

(21) Appl. No.: 18/858,369

(22) PCT Filed: Apr. 28, 2023
(86) PCT No.: PCT/US2023/020458

§ 371 Date: Oct. 21, 2024

Related U.S. Application Data
(60) Provisional application No. 63/363,871, filed on Apr. 29, 2022.

(51) Int. Cl. A61K 38/16; C12N 1/16; G01N 33/68

(52) U.S. Cl. CPC A61K 38/16; C12N 1/16; G01N 33/68; G01N 2333/40; G01N 2500/04

(57) ABSTRACT

The present disclosure provides compositions and methods for treating fungal infections. Disclosed here are proteins (Ait1p) that bind to TORC1 and regulate cell growth in yeast cells. These proteins and agents that bind to them may be effective in treating infection, particularly those caused by yeast, such as Candida glabrata.

### RELATED APPLICATION

This application is a U.S. National Stage Entry of International Patent Application No. PCT/US2023/020458 filed Apr. 28, 2023 which claims priority to U.S. Provisional Application 63/363,871 filed on Apr. 29, 2022, the contents of which are incorporated herein by reference in their entireties for all purposes.

### GOVERNMENT RIGHTS

This invention was made with government support under Grant Nos. GM097329 and T32GM136536 awarded by National Institutes of Health. The government has certain rights in the invention.

### SEQUENCE LISTING

The instant application contains a Sequence Listing which has been submitted via Patent Center and is hereby incorporated by reference in its entirety. Said .xml copy, created on Jun. 16, 2023 is named P2007WO_00582003, and is 11,474 bytes in size.

### BACKGROUND

According to the Centers for Disease Control (CDC), there were 75,000 hospitalizations and nearly 9 million outpatient visits caused by fungal infections in the U.S. in 2014, costing the country >11 billion dollars. At least ⅓ of these hospital visits are caused by Candida yeasts, and Candida infections kill 2000-4000 people in the US, and over 300,000 people in the world, each year. Moreover, these numbers are likely to rise.

One of the hallmarks of yeast is their ability to adapt to, and grow in, a wide range of environments. Most yeasts can grow on numerous carbon and nitrogen sources and enter a highly stress and starvation resistant quiescent state upon starvation. Yeast goes through many growth, stress and starvation steps during an infection cycle (from surviving on hospital surfaces and equipment to surviving when immune cells attack and engulf them). Disrupting these growth transitions can block infections.

To function correctly, cells set their growth rate based on a wide array of factors, such as nutrient, stress, and hormone signals. In eukaryotes, this fine-tuned control depends, in a large part, on a single, highly conserved, signaling hub called the Target of Rapamycin kinase Complex I (TORC1)¹⁻³. In the presence of pro-growth hormones and abundant nutrients, TORC1 drives growth by activating protein, ribosome, lipid, and nucleotide synthesis¹⁻¹¹. In contrast, when nutrient or hormone levels drop, TORC1 is repressed, causing cells to switch from anabolic to catabolic metabolism, and eventually enter a quiescent state^{12,13}.

There is a need for more effective agents and/or methods to treat yeast infection, for instance, Candida infection, and more particularly, multidrug-resistant strains.

### SUMMARY

The present disclosure provides compositions and methods that help solve the problems outlined above.

In one embodiment, it is disclosed that the Ait1 protein regulates cell growth and metabolism via TORC1. In one aspect, Ait1 is present in certain eukaryotes (e.g., yeast) that cause infection in humans, but is not present in human cells. In another aspect, Ait1 is a GPCR-like protein that binds to TORC1 in yeast and can be used to manipulate cell growth.

In another embodiment, methods of screening for a candidate compound effective in treating fungal infection are disclosed. The method may include (a) contacting said plurality of compounds with Ait1 protein or fragment thereof; and (b) selecting the candidate compound that binds to the Ait1 protein or fragment thereof or alter retention of TORC1 by Ait1. In one aspect, the readout in step (b) is altered localization of TORC1.

In another embodiment, compositions for treating or preventing fungal infection are disclosed. Agents obtained in the screening described above may be used as agent for treating or preventing fungal infection.

In another embodiment, compositions for treating or preventing fungal infection are disclosed. The compositions may comprise an engineered Ait1 protein or fragment thereof. In one aspect, the engineered Ait1 protein or fragment thereof binds to TORC1 with an altered affinity than wildtype Ait1 protein from same strain. In another aspect, the engineered Ait1 protein or fragment thereof binds to TORC1 with a higher affinity than wildtype Ait1 protein from same strain. In another aspect, the engineered Ait1 protein or fragment thereof comprises one or more mutations. In another aspect, these one or more mutations causes the changes in affinity, either increased or decreased.

In another embodiment, the disclosed compositions may be administered to a subject to treat fungal infection alone or along with pharmaceutically acceptable carriers.

In one embodiment, the subject has contracted fungal infection caused by Candida glabrata. In another embodiment, the subject has been treated with drugs for fungal infection but the Candida glabrata has become drug resistant.

In another embodiment, the disclosed agents or the disclosed engineered Ait1 protein or fragment thereof slow down growth of the Candida glabrata in the subject.

In another embodiment, the disclosed agent binds to an Ait1 protein or fragment thereof wherein the Ait1 protein or fragment thereof is endogenous to the Candida glabrata strain.

In another embodiment, the Candida glabrata strain is pathogenic. In one aspect, the Candida glabrata strain is resistant to drug treatment. In another aspect, the Candida glabrata strain is resistant to multiple drugs.

In another embodiment, the disclosed composition or agents are administering to the subject. Administration may be through a number of forms, including but not limited to oral, topical, inhale or through injection into the blood.

In another embodiment, a method of modulating growth of a yeast cell having an endogenous Ait1 gene is disclosed. The method comprising a) adding an agent to a culture comprising said yeast cell, said agent binding to the endogenous Ait1 gene in the yeast cell, and b) allowing said agent to enter said yeast cell and modulate growth rate of said yeast cell. In one aspect, the agent slows down growth rate of said yeast cell by at least 50%, or 60%, or 80%, or 90%, or 95% as compared to growth rate of same strain without the agent. In another aspect, the agent slows down growth rate of said yeast cell by 100%, essentially stopping its growth.

In another embodiment, the agent binds to the endogenous Ait1 gene in the Candida glabrata.

In another embodiment, the cell culture is a yeast cell culture and contains an engineered yeast cell that that is engineered to produce a chemical or a non-native protein. In one aspect, the agent reduces the growth rate of the yeast cell and increases production of the chemical or the non-native protein, for example, an antibody.

In another embodiment, a method of modulating growth of a yeast cell having an endogenous Ait1 gene is disclosed, said method comprising introducing one or more mutations into the endogenous Ait1 gene to generate an engineered yeast cell, said one or more mutations causing the engineered yeast cells to grow faster or slower than a wildtype yeast cell with the same genetic background other than the one or more mutations in the Ait1 gene.

### BRIEF DESCRIPTION OF THE DRAWINGS

FIG. 1 shows the TORC1 interactome in budding yeast. Blue circles show the number of background corrected Peptide Spectral Maps (PSMs) from each protein identified in a specific Kog1 or Pib2 immunopurification, while the red to yellow scale shows the average number of PSMs across all experiments. The figure shows data for the top 45 TORC1 interactors (those identified in seven or more immunopurifications).

FIG. 2 (SEQ ID NO: 2) shows Ait1 as a putative seven-helical transmembrane (GPCR-like) protein that localizes to the vacuolar membrane. (a) The predicted topology of Ait1/Ydl180w from Protter 1.0⁷³. The two large cytosolic loops in Ait1, both of which are predicted to be intrinsically disordered (C3 and C4), are labelled. (b, c) GFP-Ait1 localizes to the vacuolar membrane, as shown by the overlap between the GFP-Ait1 signal and the vacuolar membrane stain FM4-64 signal and does not relocalize in amino acid starvation (shown), or other starvation conditions (not shown).

FIG. 3 shows the Ait1 Interactome. Blue circles show the number of background corrected Peptide Spectral Maps (PSMs) for the top 25 proteins identified in the GFP-Ait1 immunopurification (based on the average number of PSMs in the four experiments), along with the data for Gtr1, Gtr2 and Pib2 for comparison.

FIG. 3s shows TORC1 interacts with Ait1. Immunopurification experiments, carried out after treatment with the DSP crosslinker, confirm that Ait1 interacts with TORC1 in vivo. (a) GFP-Ait1 co-purifies with Kog1-FLAG both in the presence, and absence, of amino acids. (b) GFP-Ait1 co-purifies with Kog1-FLAG even after long term amino acid and glucose starvation, but at 50% of the level found in log phase growth. Note (i) that Kog1 partially degrades during the IP and thus shows up as multiple bands and (ii) that we were unable to detect Kog1-FLAG or Ait1 after 2 hours of nitrogen starvation (three total attempts), likely due to changes in the solubility of localization of the TORC1-Ait1 complex. (c) Co-immunopurification carried out as in (a) but with 4x protease inhibitors. The interaction between Kog1-FLAG and GFP-Ait1 was not detected in the absence of the crosslinker (data not shown).

FIG. 4 shows TORC1-body formation during nitrogen starvation in strains missing key TORC1 interactors. Each square on the heat map shows the fraction of cells with a Kog1-YFP focus/body at a specific time-point, calculated by examining the images of >200 cells, per strain, per timepoint. Replicate experiments confirmed the severe defects in the syg1Δ, vps30Δ, rtg2Δ, and vnx1Δ strains (<15% bodies after 1 hr of nitrogen starvation). These follow-up experiments also revealed dramatic variation in the results for vsb1Δ cells (even comparing between colonies) leading us to drop the strain from our analysis.

FIG. 5 shows TORC1-body formation in the ait1Δ strain. (a) Kog1-YFP localization in the wild-type and ait1Δ strains, during log-phase growth in nutrient rich (SD) medium. The white bar shows 5 μm. (b) Fraction of wild-type and ait1Δ cells that form Kog1-YFP foci during nitrogen starvation. The points and error bars show the average and standard deviation from five replicate experiments, with at least 100 cells examined, per time point, per replicate. (c) Impact of the Ait1 deletion on TORC1-body formation in the wild-type and various mutant strains (compare left and right columns). Experiments were carried out in at least duplicate with over 200 cells examined per time point, per mutant. Individual timepoints have errors ranging from 0.05 to 0.10.

FIG. 5s shows Gtr1 and Pib2 localization in the ait1Δ strain. (upper panel) Gtr1-YFP and GFP-Pib2 localization in the wild-type and ait1Δ strains, during log phase growth in nutrient rich (SD) medium and one hour of complete nitrogen starvation (-N). The white bar in the differential interference contrast image (left column) shows 5□m; fluorescence images are on the right. (lower panel) Fraction of wild-type and ait1Δ cells that form Gtr1-YFP and GFP-Pib2 foci during nitrogen starvation. The points and error bars show the average and standard deviation from three replicate experiments, with at least 200 cells examined, per time point, per replicate.

FIG. 6 shows impact of Ait1 on TORC1 activity during amino acid starvation. (a) TORC1 activity during complete amino acid starvation (top), and leucine starvation (bottom), in wild-type, ait1Δ, and GTR1^{Q65L} (Gtr1^{on}) strains, as measured by Western blot using an anti phospho-Rps6 antibody. (b) Values show the ratio of the p-Rps6 signal divided by the PGK (loading control) signal in each lane, relative to the value for the wild-type strain at time=0. Wild-type and mutant strains were grown and processed together and run on the same gel.

FIG. 6s shows Impact of Ait1 on TORC1 activity during nitrogen and amino acid starvation. (a) TORC1 activity during complete nitrogen starvation in wild-type and ait1Δ strains, as measured by Western blot using an anti phospho-Rps6 antibody. Graphs show the ratio of the p-Rps6 signal divided by the PGK (loading control) signal in each lane, relative to the value for the wild-type strain at time=0. Wild-type and mutant strains were grown and processed together and run on the same gel. (b) Mobility shift assay following the phosphorylation of Sch9-HA during leucine starvation on a Western blot.

FIG. 7 shows impact of Ait1 on TORC1 activity in strains with mutations in Gtr1/2, or the prion domains in Kog1, during amino acid starvation. (a) TORC1 activity during complete amino acid starvation in mutant strains with (left column) and without Ait1 (right column), measured using a Western blot, as described in FIG. 6. (b) Values show the ratio of the p-Rps6 signal divided by the PGK (loading control) signal in each lane, relative to the value for the wild-type strain at time=0. Mutant and double mutant strains were grown and collected together and run on the same gel.

FIG. 8 shows the role of the Ait1 C3 loop in TORC1 regulation. (a) (FIG. 8A SEQ ID NO: 3-4) BLAST alignment of the SLC38A9 (top) and Ait1 sequences, showing the entire C3 loop (no other sequences in these proteins align). (b, c) (FIG. 8C SEQ ID NO: 5-8) TORC1 activity in Ait1 C3 and C4 loop mutants during leucine starvation, measured as described in FIG. 6. Values show the ratio of the p-Rps6 signal divided by the PGK (loading control) signal in each lane, relative to the value for the wild-type strain at time=0. Mutant and wild-type strains were grown and collected together and run on the same gel.

FIG. 8s shows localization of Ait1 and the Ait1 C3 and C4 loop mutants. Images taken of strains expressing GFP-Ait1, GFP-AitΔC3, GFP-AitΔC4, GFP-Ait1C3v1, GFP-Ait1C3v2, and GFP-Ait1C3v3 show that the mutants fold and are transported to the vacuolar membrane correctly. Note, however, that the abundance of GFP-AitΔC3 is decreased to approximately 65% of that found in the wild type (values from line-plot analysis of ten cells in each image). The white bar shows 5 μm.

FIG. 9 shows Impact of Alt1 C3 and C4 loop mutations on TORC1 localization and Gtr1/2 binding. (a) Each square on the heat map shows the fraction of cells with a Kog1-YFP focus/body at a specific time-point (as labelled), calculated by averaging the data from three replicate experiments (>100 cells analyzed at each time-point and replicate). Individual timepoints have errors ranging from 0.02 to 0.12 (average 0.06). (b) Bar graph showing the fraction of cells with a TORC1-body during log phase growth in SD medium. (c). Co-immunoprecipitation showing a strong interaction between GFP-Ait1 and Gtr1-myc, but not GFP-Ait1C3v2 and Gtr1-myc. The graph shows the ratio of the Gtr1 and Ait1 signals in the wild-type (black line) and Ait1C3v2 (blue line) strains, before, and 30 min after, amino acid starvation.

FIG. 10 shows evolution of the TORC1 circuit in yeast. Species identified as carrying Ait1 in a BLAST search (p<0.001 cutoff) are marked with a red circle on a previously constructed map of Rheb and TSC1/2 conservation among the budding yeast, taken from Tatebe and Shiozaki³⁶. The light blue circles denote the presence of a highly divergent (non-functional) Rheb in species closely related to S. cerevisiae³⁶. Ait1 was not detected in any of the yeasts outside the Saccharomycesceae and Saccharomycodaceae.

FIG. 11 (SEQ ID NO: 9) shows Coding sequence of Ait1 in Saccharomyces cerevisiae.

### DETAILED DESCRIPTION

The present disclosure provides compositions and methods for treating infections. In one embodiment, it is disclosed here that the Ait1 protein regulates cell growth and metabolism via TORC1. In one aspect, Ait1 is present in certain eukaryotes (e.g., yeast) that cause infection in humans, but is not present in human cells. In another aspect, Ait1 is a GPCR-like protein that binds to TORC1 in yeast and can be used to manipulate cell growth.

In one embodiment, Ait1 can be mutated to manipulate the growth of yeast cells used in manufacturing or other bioengineering processes. In another embodiment, Candida glabrata has Ait1 and therefore, mutant Ait1 or agents (e.g., small molecule chemicals) that bind to Ait1 may be used to slow the growth of Candida glabrata, the second deadliest fungal pathogen.

TORC1 is regulated by a sophisticated signaling network that, in humans, includes two well defined channels: (I) Growth factor and mitogen signals are transmitted to TORC1 through a GTPase Activating Protein (GAP) called the Tuberous Sclerosis Complex (TSC)^{14,15}. In the presence of pro-growth hormones (such as insulin), TSC is repressed, triggering accumulation of the active, GTP-bound, form of Rheb^{16,17}. GTP-Rheb then binds to TORC1 on the lysosomal membrane, driving a conformational change that increases TORC1 activity^{16,18}. The AMP activated protein kinase (AMPK) also signals to TORC1 via TSC (as well as the TORC1 subunit Kog1/Raptor) to ensure TORC1 is inhibited when ATP levels fall¹⁹⁻²¹. (II) Amino acid (and glucose) signals are transmitted to TORC1 via a heterodimeric pair of GTPases, consisting of RagA or B and RagC or D, that are tethered to the lysosomal membrane by the Regulator complex²²⁻²⁶. In the presence of adequate nutrients, RagA/B and C/D are in their GTP and GDP bound forms, respectively, and bind tightly to TORC1 to keep it on the lysosomal membrane and near Rheb^{16,22-24,27}. However, when amino acid levels fall, the large multiprotein GAP, GATOR1/2, drives RagA/B to the GDP bound form, triggering the release of TORC1 from the lysosome so that it cannot be activated by Rheb²⁸.

GATOR1/2, in turn, is regulated by at least three different amino acid binding proteins to ensure that cell growth halts during starvation: the leucine sensor Sestrin2^{29,30}; the arginine sensor CASTOR1^{31,32}; and the methionine—or more specifically S-adensylmethionine (SAM)—sensor SAMTOR³³. Arginine signals are also transmitted to the Rags via SLC38A9, an amino acid transporter in the lysosomal membrane^{34,35}.

Outside of humans, however, much less is known about TORC1 regulation. The amino acid sensors discussed above are only fully conserved in vertebrates^{3,36}, and while Rheb/TSC and the Rags/GATOR are ancient TORC1 pathway components—likely present in the last common eukaryote—many yeasts, worms, plants, and protists/excavata have lost Rheb and TSC^{3,36}.

One well studied organism with a TORC1 signaling network that appears to have diverged significantly from that in humans is the budding yeast, Saccharomyces cerevisiae. S. cerevisiae has two GTPases, Gtr1 and Gtr2, that are homologs of RagA/B and RagC/D, respectively^{37,38}. Furthermore, Gtr1/2 are tethered to the vacuole (the yeast equivalent of the lysosome) by a complex that is very similar—but not obviously homologous to—the Ragulator, called Ego1, Ego2 and Ego3³⁹⁻⁴¹. The GATOR1/2 GAP that acts upstream of the Rags is also conserved in yeast, and made up of Npr2, Npr3 and Iml1 (the GATOR1 equivalent, known as SEACIT) and Rtc1, Mtc5, Sea4, Seh1 and Sec13 (the GATOR2 equivalent, known as SEACAT)⁴²⁻⁴⁶. However, S. cerevisiae do not have SLC38A9, Sestrins, CASTOR or SAMTOR, and are also missing TSC1/2 and functional Rheb^{1,3,36}.

In line with the expectation that there are differences between TORC1 signaling in yeast and humans, it was recently discovered that glucose and nitrogen starvation cause TORC1 in S. cerevisiae to move from its position distributed around the vacuolar membrane to a single body on the edge of the vacuole^{47,48}. Adding the missing nutrient back to the cell—even in the presence of cycloheximide—then reverses the process⁴⁷. It has also been found that TORC1-body formation is initiated by inactivation of Gtr1/2, and requires an interaction between TORC1 and the recently identified TORC1 regulator Pib2⁴⁷⁻⁵³. TORC1 agglomeration, itself, is then driven by two glutamine-rich, prion-like domains in the TORC1 subunit Kog1/Raptor, and ultimately functions to increase the threshold for TORC1 reactivation⁴⁷. In other words, the formation of TORC1-bodies helps to ensure that cells commit to the quiescent state when they have been starving for a significant period of time. Interestingly, the prion-like domains in Kog1/Raptor are found in yeast species and worms that are missing the TSC genes, but are absent from S. pombe and higher organisms that do carry the TSC genes⁴⁷. This suggests that organisms use either TSC and Rheb, or TORC1-body formation, alongside Gtr1/2 (Rag proteins) to control TORC1 activity⁴⁷.

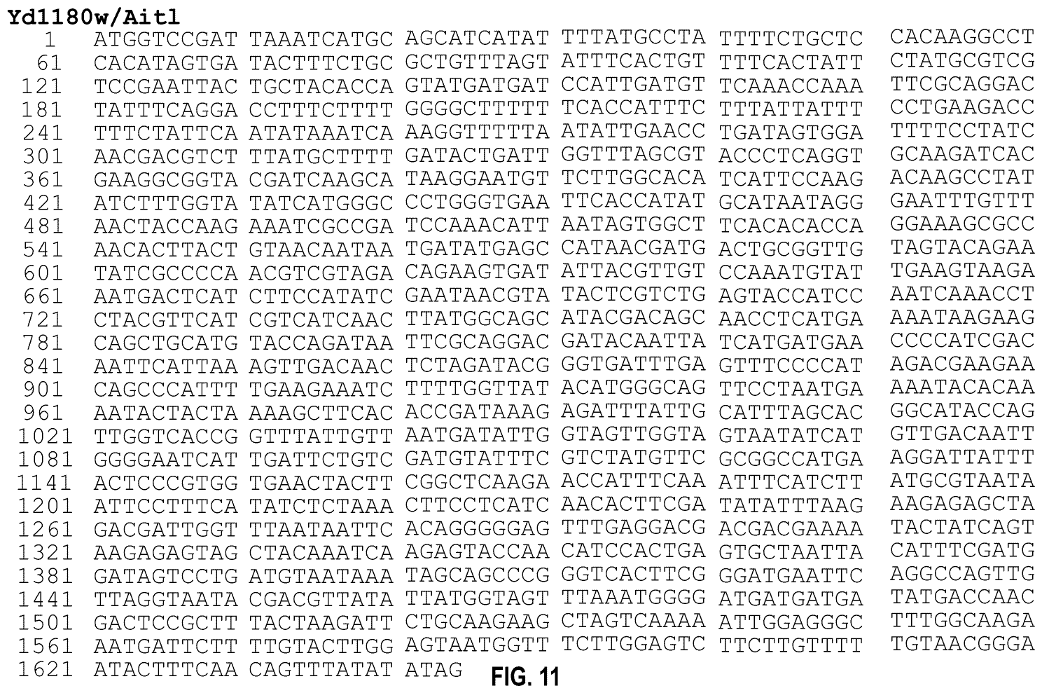

In this disclosure, the TORC1 interactome in S. cerevisiae is mapped in a wide range of stress and starvation conditions. These experiments lead to the identification of numerous new TORC1 regulators, the most notable of which are a putative phosphate channel, Syg1, and a previously unstudied GPCR-like protein, Ydl180w, named Ait1 (Amino acid dependent Inhibitor of TORC1). The coding sequence of Ait1 is shown in FIG. 11. In follow-up experiments, it is shown that Ait1 is required to hold TORC1 in its native position around the vacuolar membrane during log-phase growth. It is also shown that Ait1 is required for TORC1 inhibition during amino acid starvation, and helps to drive Gtr1/2 from their GTP/GDP bound (active) form, to their GDP/GTP bound (inactive) form. Ait1 is only found in the Saccharomycesaceae and Saccharomycodaceae. The yeast species within these related families—which include the pathogen Candida glabrata—are unique in that they (i) have highly divergent Rheb, or no Rheb, (ii) are missing TSC2 and/or TSC1, and (iii) have prion-like domains in the TORC1 subunit Kog1/Raptor³⁶. Thus, an ancestor of the Saccharomycesaceae/codaceae gained the novel TORC1 regulator, Ait1, at around the same time it lost functional Rheb and TSC1/2 (approximately 200 million years ago^{36,54}), to aid in amino acid signaling and appropriate TORC1 localization. Similar rewiring of the TORC1 pathway likely occurred during the evolution of many other simple eukaryotes. Ait1 represents an important new drug target in yeast.

In certain embodiments, it is shown here that: (i) Ait1, binds to TORC1 and holds it around the vacuolar membrane during log-phase growth; (ii) Ait1 acts through Gtr1/2 (most likely Gtr2) to inhibit TORC1 during amino acid starvation; and (iii) Ait1 regulates TORC1 via a central region in its 180 amino acid C3 loop, that resembles the Rag A/C (Gtr1/2) binding domain in SLC38A9.

In one embodiment, one model to explain the data: First, at the onset of amino acid starvation, SEAC is activated and triggers GTP hydrolysis in Gtr1. Next, the resulting conformational change in Gtr1, and/or signals transmitted through Ait1, promote binding of the C3 loop in Ait1 to Gtr1/2. Finally, Ait1 drives the release of GDP from Gtr2, completing the conversion of Gtr1/2 from the active GTP/GDP-bound state, to the inactive GDP/GTP-bound state.

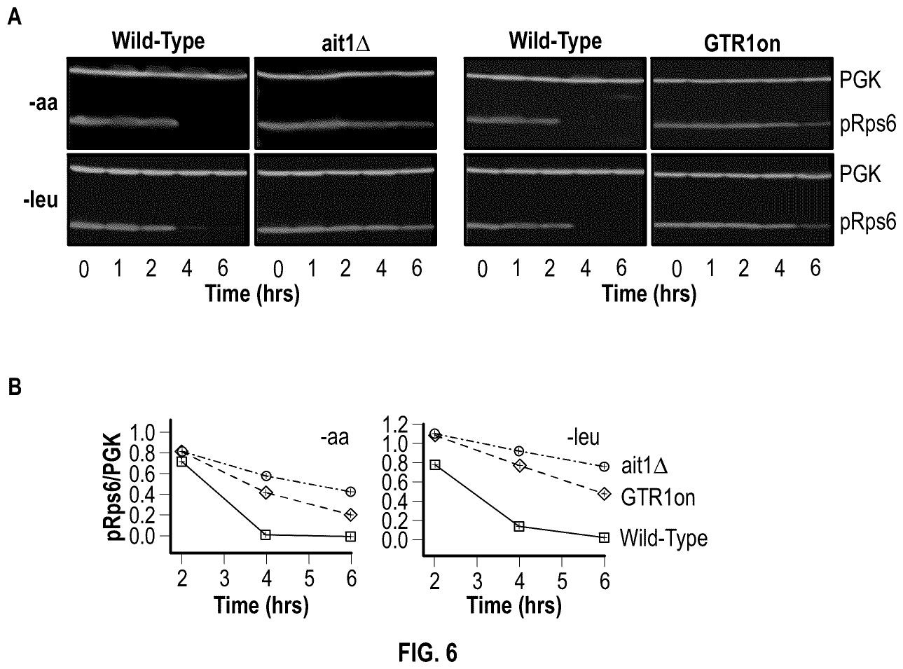

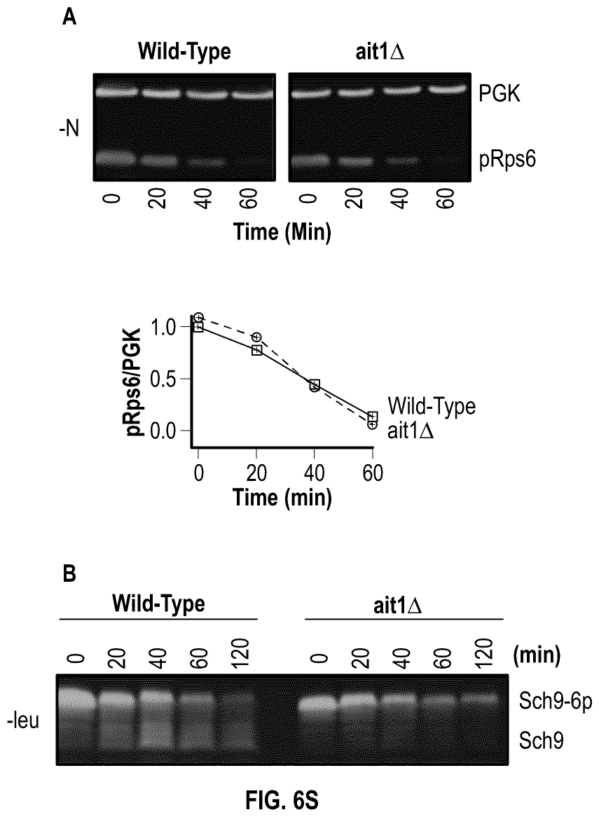

In another embodiment, as an extension of the above model, it may be that strong signals through SEAC, such as those in complete nitrogen starvation (FIG. 6s), override the need for Ait1 due to coupling between the GTP binding sites in Gtr1 and Gtr2⁶⁷.

In another embodiment, while this model provides some explanation for all of the available data, it remains unclear how exactly Ait1 regulates Gtr1/2; Ait1 could also promote TORC1 inhibition by stabilizing the inactive state of Gtr1/2, controlling access to Gtr1/2 activators/repressors, and/or other related mechanisms.

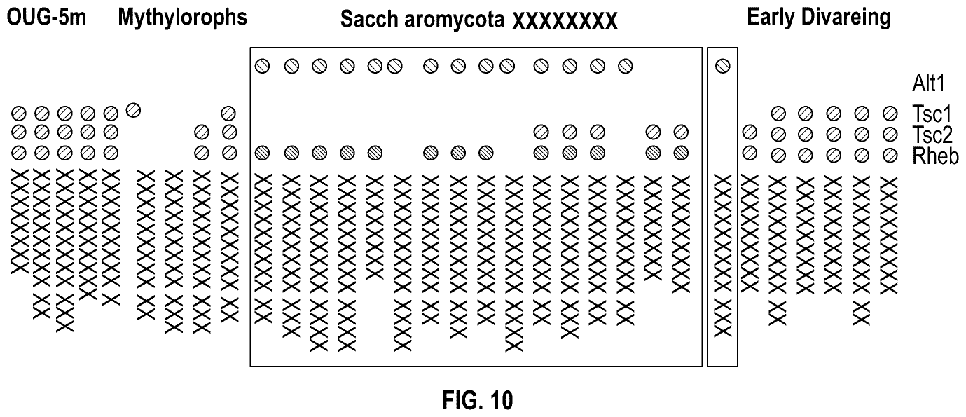

The dual role of Ait1 in holding TORC1 around the vacuolar membrane and helping to regulate TORC1 via Gtr1/2 is especially interesting in the context of yeast evolution. Ait1 is found in species throughout the closely related Saccharomycesceae and Saccharomycodaceae, but not in other clades (Table S2). The Saccharomycesceae/codaceae, which include Saccharomyces cerevisiae, Ashbya gossypii, Kluyveromyces lactis, and the pathogen Candida glabrata, split from other yeasts approximately 200 million years ago⁵⁴, and are unique in that they have highly divergent Rheb, or no Rheb, and have lost TSC2 and/or TSC1 (FIG. 10). They are also unique in that many species in these families have prion-like, glutamine rich, domains in Kog1/Raptor; domains that at least in S. cerevisiae help control the commitment to quiescence⁴⁷. Thus, a common ancestor of the Saccharomycesceae/codaceae lost functional Rheb and TSC1/2, gained prion-like domains in Kog1, and gained Ait1.

In another embodiment, it is likely that these events were linked. First, the data presented here shows that Ait1 is required to block TORC1 from agglomerating via its prion-like domains during log-phase growth in nutrient replete conditions. That is, without Ait1 dependent anchoring of TORC1, the addition of prion-like domains in TORC1 would have triggered constitutive body formation, rather than add a reversible, regulatory, transition to the TORC1 circuit. Second, our data shows that Ait1 has taken over part of the role of ancestral Rheb⁶⁸ by helping to regulate TORC1 in starvation conditions.

In another embodiment, it remains uncertain how Gtr1/2 regulate TORC1 in the absence of Rheb. It is likely that while the core of the TORC1 pathway (including TORC1itself and Gtr1/2) is highly conserved, other aspects of this ancient growth control circuit are plastic and have changed significantly during evolution. Learning more about these changes will not only shed light on the way that eukaryotes have adapted to different niches, it should also open the door to creating drugs that specifically block the growth of fungal pathogens and a variety of parasites.

In another embodiment, a composition for treating a fungal infection is disclosed. The composition may contain an engineered Ait1 protein or fragment thereof designed to bind to TORC1 and/or Gtr1/2 with an altered affinity as compared to wildtype Ait1 protein. In one aspect, the engineered Ait1 protein or fragment thereof binds to TORC1 with a higher affinity than wildtype Ait1 protein. In one aspect, the engineered Ait1 protein or fragment thereof may contain one or more point mutations, deletions or additions.

In another embodiment, a method is disclosed for treating a fungal infection in a subject, which includes delivering an agent to the subject, wherein the agent binds to Ait1 protein or fragment thereof. The agent may be a small molecule chemical, a protein, a polynucleotide, or an antibody or other therapeutic agents.

In another embodiment, the fungal infection may be an infection caused by Candida glabrata. Candida glabrata contains the Ait1 protein which may be a target for a therapeutic agent. In one aspect, a therapeutic agent that binds to fungal Ait1 is advantageous because human does not have an Ait1 homolog. In another embodiment, the therapeutic agent binds to fungal Ait1 but does not bind to a cellular target in human.

In another embodiment, a method is disclosed for modulating growth of a yeast cell having an endogenous Ait1 gene. The method may include introducing one or more mutations into the endogenous Ait1 gene to generate an engineered yeast cell, wherein the one or more mutations cause the engineered yeast cells to grow faster or slower than a wildtype yeast cell with an otherwise identical genetic background (i.e., the only difference in genetic background is in those mutations on the Ait1 gene).

In another embodiment, in bioengineering where cells are cultured to produce chemicals or biologics of interest, an agent can be added to the culture to stop growth of the cells and force the cells to produce only the substance of interest. In one aspect, the agent binds to Ait1. In another aspect, the agent modulates Ait1 and stops growth of the yeast cells. In another aspect, the agent is added to the culture, and at the same time, nutrient supply to the cell culture is restricted to regulate growth of the cells.

In another embodiment, a method is disclosed for modulating growth of a yeast cell having an endogenous Ait1 gene, the method may include (a) adding an agent to a culture comprising said yeast cell, wherein the agent binds to the endogenous Ait1 gene in the yeast cell, and (b) allowing said agent to enter the yeast cell and modulate growth rate of the yeast cell.

In one aspect, the agent may be one that exist in nature. In another aspect, the agent may be one that is synthesized in a lab. In another embodiment, a method of screening for such an agent is disclosed. The method may include: (a) contacting a plurality of candidate compounds with Ait1 protein or fragment thereof; and (b) selecting the candidate compound that binds to the Ait1 protein or fragment thereof or selecting the candidate compound that alters retention of TORC1 by Ait1. In another embodiment, the readout in step (b) is altered localization of TORC1. By way of example, an agent may bind to Ait1 and release TORC1 from the vacuole.

The articles “a,” “an” and “the” are used to refer to one or more than one (i.e., to at least one) of the grammatical object of the article.

The terms “comprise”, “comprising”, “including” “containing”, “characterized by”, and grammatical equivalents thereof are used in the inclusive, open sense, meaning that additional elements are not expressly mentioned but may be included. It is not intended to be construed as “consists of only.”

The term “subject” or “patient” as used herein is intended to include animals. Examples of subjects include but are not limited to mammals, e.g., humans, apes, monkeys, dogs, cows, horses, pigs, sheep, goats, cats, mice, rabbits, rats, and transgenic non-human animals. In an embodiment, the subject is a human.

The term “biological sample” or “sample” encompasses a variety of sample types obtained from an organism. The term encompasses bodily fluids such as blood, saliva, serum, plasma, urine and other liquid samples of biological origin, and solid samples, such as a nasopharyngeal swab, a biopsy specimen or tissue cultures or cells derived therefrom and the progeny thereof.

The terms “polypeptide,” “peptide” and “protein” may be used interchangeably in this disclosure. The terms “oligonucleotide,” and “polynucleotide” may also be used interchangeably in this disclosure.

The present disclosure can be further illustrated by the following items:

Item 1. A composition for treating a fungal infection, comprising an engineered Ait1 protein or fragment thereof, said engineered Ait1 protein or fragment thereof binding to TORC1 with an altered affinity than wildtype Ait1 protein from same strain.

Item 2. The composition of Item 1, wherein the engineered Ait1 protein or fragment thereof binds to TORC1 with a higher affinity than wildtype Ait1 protein from same strain.

Item 3. The composition of any of preceding items, wherein the engineered Ait1 protein or fragment thereof comprises one or more mutations.

Item 4. A method for treating a fungal infection in a subject in need thereof, comprising administering to the subject the composition of Item 1.

Item 5. The method of Item 4, wherein the subject has contracted fungal infection caused by Candida glabrata.

Item 6. The method of any of Items 4 and 5, wherein the engineered Ait1 protein or fragment thereof slows down growth of the Candida glabrata in the subject.

Item 7. A composition for treating a fungal infection, comprising an agent that binds to Ait1 protein or fragment thereof.

Item 8. The composition of Item 7, wherein said agent binds to an Ait1 protein or fragment thereof endogenous to a pathogenic Candida glabrata strain.

Item 9. A method for treating a fungal infection in a subject, comprising administering to the subject the composition of Item 7.

Item 10. The method of Item 9, wherein the subject has contracted fungal infection caused by Candida glabrata.

Item 11. The method of any of Items 9 and 10, wherein the Candida glabrata is resistant to drug treatment.

Item 12. The method of any of Items 9-11, wherein the agent slows down growth of the Candida glabrata in the subject.

Item 13. A method of modulating growth of a yeast cell having an endogenous Ait1 gene, said method comprising
a) adding an agent to a culture comprising said yeast cell, said agent binding to the endogenous Ait1 gene in the yeast cell, and
b) allowing said agent to enter said yeast cell and modulate growth rate of said yeast cell.

Item 14. The method of Item 13, wherein the agent slows down growth rate of said yeast cell.

Item 15. The method of any of Items 13 and 14, wherein the agent binds to the endogenous Ait1 gene.

Item 16. The method of any of Items 13-15, wherein the yeast cell is an engineered yeast cell that that is engineered to produce a chemical or a non-native protein.

Item 17. The method of any of Items 13-16, wherein the agent reduces the growth rate of the yeast cell and increases production of the chemical or the non-native protein.

Item 18. A method of modulating growth of a yeast cell having an endogenous Ait1 gene, said method comprising introducing one or more mutations into the endogenous Ait1 gene to generate an engineered yeast cell, said one or more mutations causing the engineered yeast cells to grow faster or slower than a wildtype yeast cell with the same genetic background other than the one or more mutations in the Ait1 gene.

Item 19. A method of screening for a candidate compound effective in treating fungal infection, comprising
a) contacting said plurality of compounds with Ait1 protein or fragment thereof; and
b) selecting the candidate compound that binds to the Ait1 protein or fragment thereof or alter retention of TORC1 by Ait1.

Item 20. The method of Item 19, wherein readout in step (b) is altered localization of TORC1.

All references cited in this disclosure, including but not limited to patents, patent applications and published papers, are hereby incorporated by reference into this disclosure.

### EXAMPLES

The disclosure will now be illustrated with working examples, and which is intended to illustrate the working of disclosure and not intended to restrict any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

### Example 1 The TORC1 Interactome in Budding Yeast

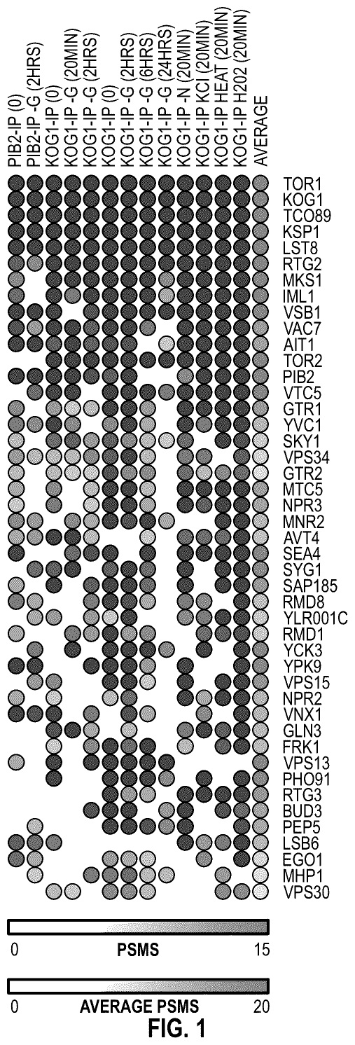

As a first step towards building a map of the TORC1 regulatory network, an immunopurification protocol was developed which makes it possible to capture and identify TORC1 interactors. Cells carrying Kog1-FLAG, and in parallel cells carrying Kog1-HA, were grown to log-growth phase, or grown to log-growth phase and transferred into stress or starvation medium, rapidly filtered and flash frozen. The cells were then lysed and treated with the short (12 Å) cleavable crosslinker dithiobis (succinimidyl propionate) (DSP) and the nonionic detergent digitonin⁵⁵. The supernatants from the Kog1-HA and Kog1-FLAG strains were then immunopurified in parallel on anti-FLAG columns, the crosslinkers broken, and the samples analyzed using mass spectrometry. Proteins with at least 2-fold higher abundance in the true IP (Kog1-FLAG) versus the mock IP (Kog1-HA), and with at least 7 peptide spectral maps in the true IP, were scored as potential interactors. In total, Kog1 was immunopurified in 11 different experiments (in nine conditions). A similar experiment was conducted to identify Pib2 interactors in two conditions. These purifications led to the identification of over 200 interactors, 138 of which were identified in four or more experiments. At the top of this list are 45 proteins identified in seven or more experiments, including: (i) all four subunits of TORC1 (blue names, FIG. 1); (ii) Gtr1, Gtr2 and Ego1, all three subunits of SEACIT, and two subunits of SEACAT (green names, FIG. 1); (iii) a variety of proteins that have been shown to play some role in TORC1 signaling previously, including several TORC1 substrates^{4,56,57} (black names, FIG. 1); and (iv) 15 proteins that have, to the best of our knowledge, not been connected to TORC1 signaling previously, or in many cases, studied at all (red names, FIG. 1). Several of these new interactors—including Ydl180w/Ait1, Vsb1 and Vtc5—form interactions with TORC1 that are as tight, or tighter, than those between TORC1 and Gtr1/2, as judged by the amount of material captured in the purification (FIG. 1).

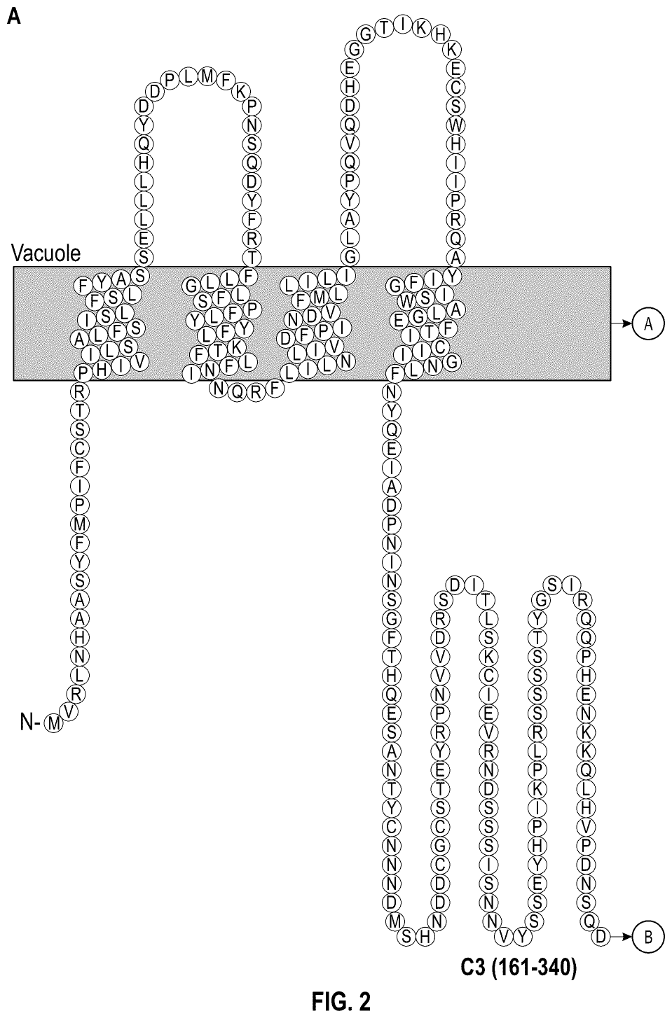

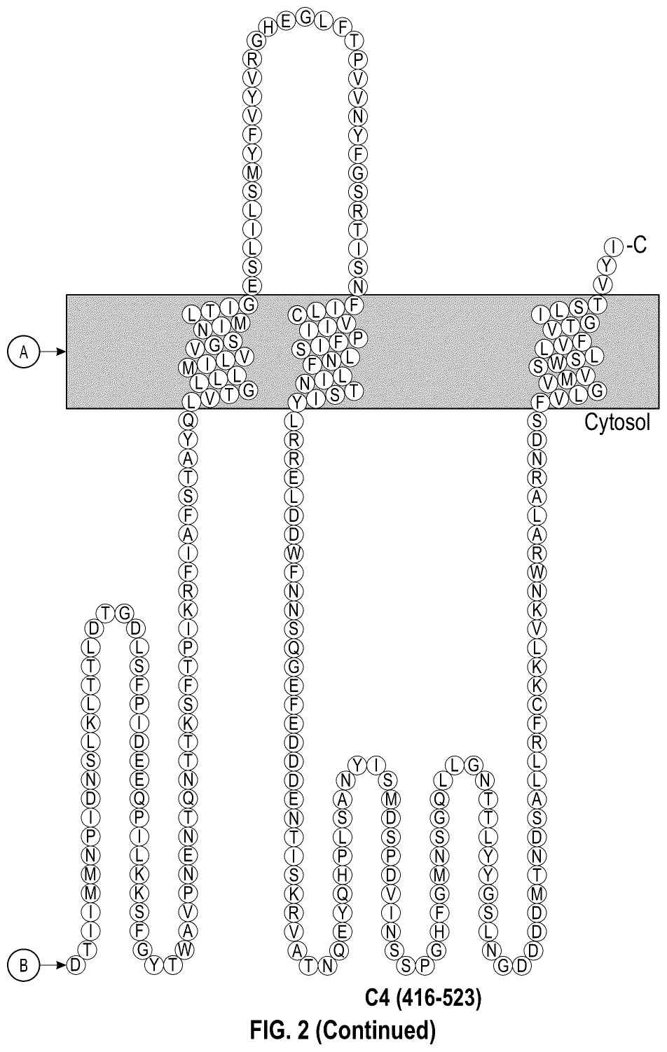

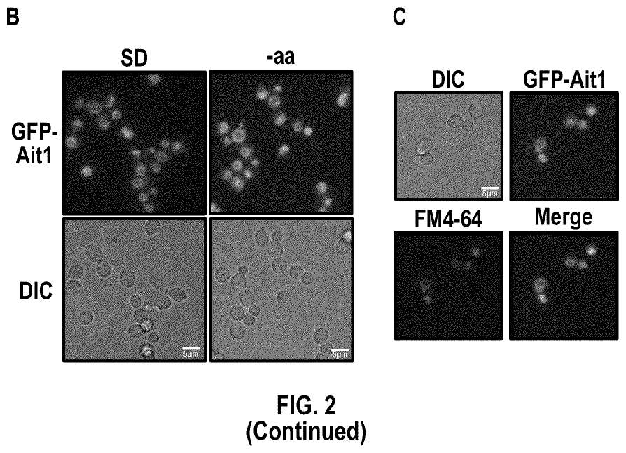

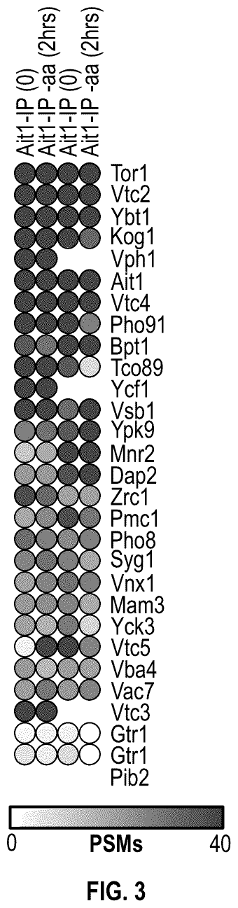

To learn more about the interaction between TORC1 and the novel interactor Ait1 (a previously unstudied GPCR-like protein located in the vacuolar membrane^{58,59}; FIG. 2), the GFP-Ait1 was also immunopurified after crosslinking, and the interactors mapped, as described above for Kog1. These experiments showed that the TORC1 subunits Kog1, Tor1 and Tco89 are among the most abundant proteins captured in an Ait1 purification, suggesting that there is a direct interaction between TORC1 and Ait1 (FIG. 3).

### Example 2 Impact of TORC1 Interactors on TORC1-Body Formation

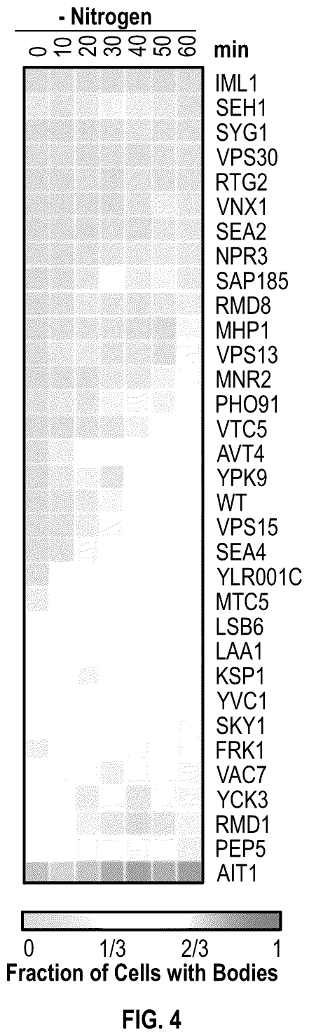

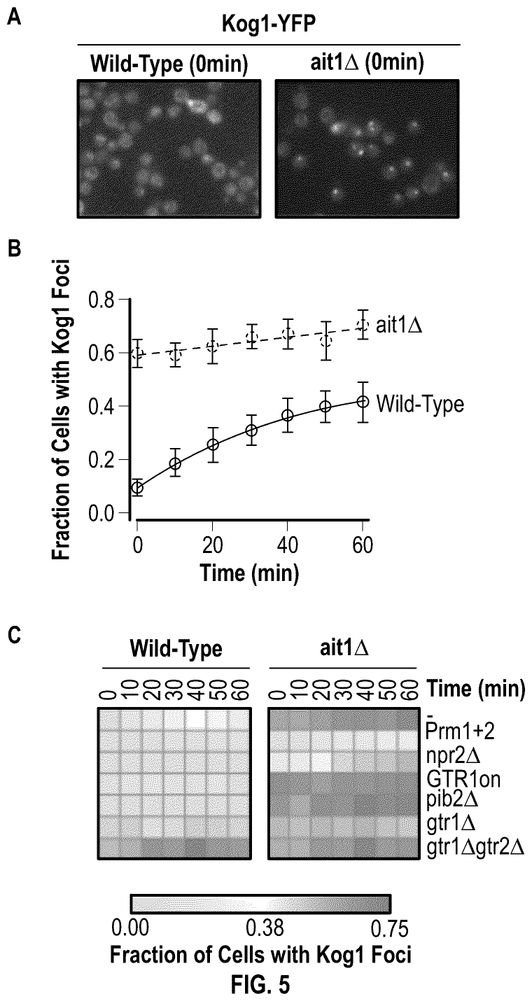

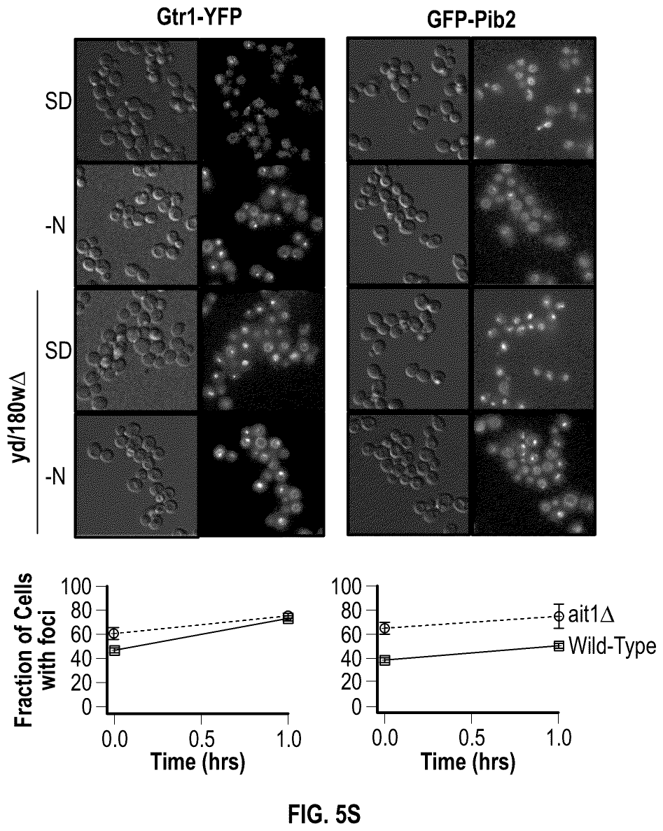

To examine the impact of the new TORC1 interactors on TORC1 signaling, Kog1-YFP localization during nitrogen starvation was studied in a collection of strains, each missing one of the top 50 proteins identified in the immunopurification experiments (excluding interactors that were examined in our previous studies^{47,48}). These experiments showed that many of the known TORC1 interactors are important for TORC1-body formation, including the SEACIT/CAT subunits Iml1, Seh1, Sea2/Rtc1, and Npr3 (FIG. 4). These experiments also showed that several of the previously unknown TORC1 interactors have a profound impact on TORC1-body formation and/or TORC1 localization. Specifically, deletion of Vnx1 (a vacuolar monovalent cation/proton antiporter⁶⁰) or Syg1 (a putative phosphate channel in the vacuolar membrane⁵⁸) blocks TORC1 body formation, just like deletion of Npr2, Npr3, Iml1 or Pib2 (FIG. 4 and ref⁴⁸). The most striking result, however, was found in the ait1D strain: Deletion of Ait1 causes TORC1 to move into a body, even during log-phase growth in nutrient replete medium (FIG. 4, FIG. 5a, b).

To learn more about Ait1 function, the impact of deleting Ait1 on Kog1-YFP localization was measured in strains carrying mutations that block, or promote, TORC1-body formation^{47,48} (FIG. 5c). These experiments revealed that deletion of Ait1 completely overrides the severe defects in TORC1-body formation caused by (i) locking Gtr1 in its active, GTP bound, conformation (GTR1^{Q65L} or Gtr1^{on} for short), (ii) deleting the Gtr1 inhibitor Npr2, or (iii) deleting of the TORC1 binding protein and regulator Pib2 (FIG. 5c). However, deletion of Ait1 does not rescue TORC1-body formation in a strain carrying Q to A mutations in the two prion-like domains of Kog1 (Prm1+2, FIG. 5c). Thus, Ait1, like Gtr1/2 (FIG. 5c), is required to hold TORC1 in its native position, distributed around the vacuolar membrane, in nutrient replete conditions. This tethering effect is then lost (or overridden) in starvation conditions.

In contrast to its influence on TORC1, Ait1 does not have a dramatic impact on the localization of the TORC1 binding proteins Gtr1/2 and Pib2, as determined by images of Gtr1-YFP and GFP-Pib2 (FIG. 5s). Instead, Pib2 and Gtr1 remain distributed around the vacuolar membrane in the ait1D strain, albeit with additional enrichment in foci on the edge of the vacuole (movement that is likely driven by TORC1 agglomeration; FIG. 5s).

### Example 3 Ait1 Inhibits TORC1 during Amino Acid Starvation

To test if Ait1 regulates TORC1 signaling, the phosphorylation of a downstream reporter of TORC1 activity, Rps6^{61,62}, was followed in wild-type and ait1D strains. These experiments showed that deletion of Ait1 almost completely blocks TORC1 repression during amino acid starvation (in a standard lab strain, FIG. 6); a phenotype similar to that seen in a strain with Gtr1 locked in its active, GTP bound, state (FIG. 6). In contrast, Ait1 does not impact TORC1 inhibition during complete nitrogen starvation (FIG. 6s).

Previous studies have shown that leucine is the primary amino acid activating TORC1 via Gtr1/2⁶³. Experiments were carried out to test if Ait1 is also required for TORC1 inhibition in cells starved for leucine. This was the case; an ait1D strain has over 80% TORC1 activity after 6 hours of leucine starvation, as judged by Rps6 phosphorylation (FIG. 6), and nearly 100% TORC1 activity as judged by the phosphorylation of the direct TORC1 substrate, Sch9 (FIG. 6s).

### Example 4 Ait1 Acts Upstream of Gtr1/2 to Regulate TORC1

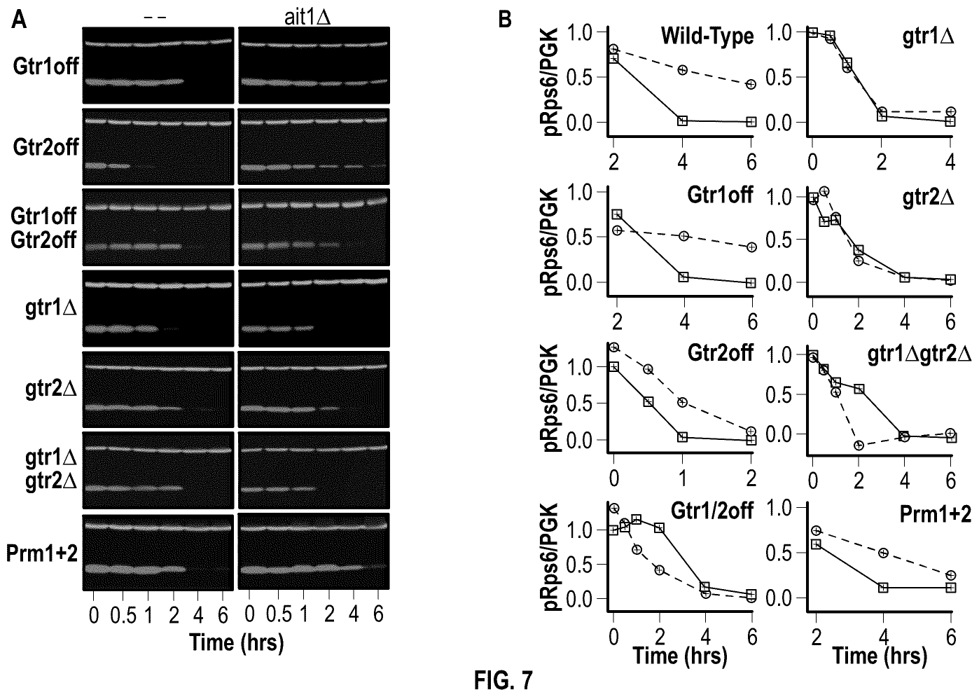

The observation that Ait1 and Gtr1/2 both regulate TORC1 during amino acid starvation led us to consider two models of Ait1 function: (i) Ait1 acts upstream of Gtr1/2 to promote TORC1 inhibition, and (ii) Ait1 acts downstream of Gtr1/2 to repress TORC1 activity once Gtr1/2 are inactivated. To distinguish between these models, the impact of Ait1 on TORC1 signaling was measured in strains with: Gtr1 locked in its GDP-bound, inactive, state (GTR1^{S20L} or Gtr1^{off} for short); Gtr2 locked in GTP-bound, inactive, state (GTR2^{Q66L} or Gtr2^{off} for short); and Gtr1 and Gtr2 both locked in their inactive states (Gtr1^{off}/Gtr2^{off})⁴². These experiments showed that Ait1 is still important for TORC1 inhibition in a Gtr1^{off} strain, but has limited impact on TORC1 inhibition in a Gtr2^{off} strain, and actually helps activate TORC1 in a Gtr1^{off}/Gtr2^{off} strain (FIG. 7). Thus, Ait1 promotes TORC1 repression via Gtr1/2 during amino acid starvation, likely by helping to drive Gtr2 into its inactive, GTP-bound, state. In line with this, deletion of Gtr1, Gtr2, or Gtr1/2 completely bypasses the need for Ait1 in amino acid starvation-dependent TORC1 signaling (FIG. 7).

### Example 5 Mechanism of Ait1 Dependent TORC1 Inhibition

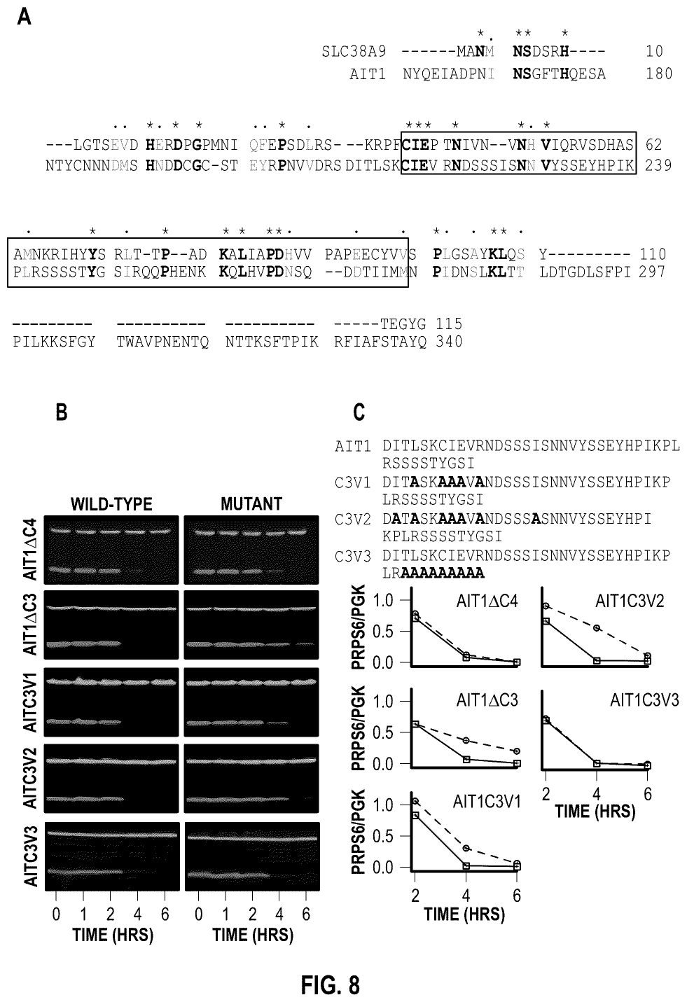

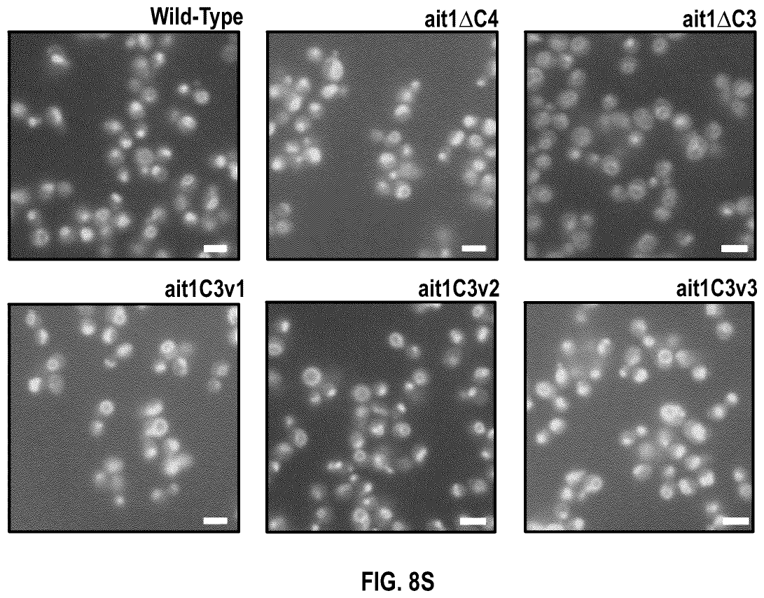

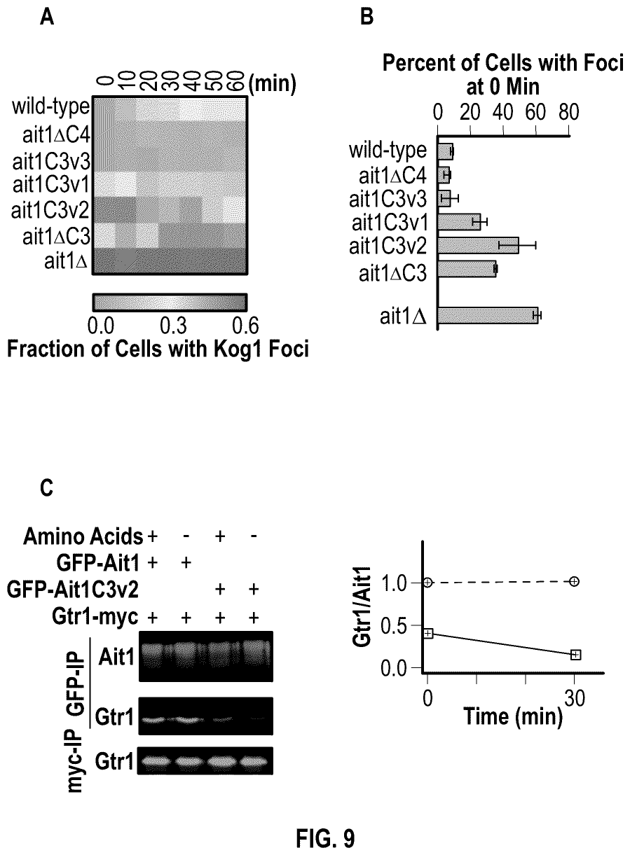

To gain insight into the mechanism underlying Ait1 dependent regulation of Gtr1/2, sequence alignments were performed to look for similarity between Ait1 and known Gtr1/2 and RagA/C binding proteins. These alignments uncovered analogous sequences in the N-terminal region of SLC38A9 and the third cytosolic (C3) loop of Ait1 (FIG. 8a). The N-terminal region of SLC38A9 has been shown to dissociate from the pore of the SLC38A9 channel in the presence of arginine, and then bind (via residues 39-97; box FIG. 8a) to a cleft at the interface between RagA and RagC^{34,64-66}. This cleft sits near the GTP binding pockets in RagA and RagC, and faces up and away from TORC1 in the RagA/C-TORC1 complex, at a distance ˜80 Å away from the vacuolar surface/membrane^{27,66}. Thus, it seemed likely that Ait1 inhibits Gtr1/2 via its 180 aa long, and intrinsically disordered, C3 loop. In line with this model, there are over 50 amino acids either side of the putative Gtr1/2 binding sequence in the C3 loop (box, FIG. 8a)—providing more than enough unstructured peptide for the C3 loop to extend over TORC1 and interact with Gtr1/2.

To test if Ait1 regulates Gtr1/2 and TORC1 via its C3 loop, two mutant versions of Ait1 were built; one in which the C3 loop, and the other in which the C4 loop, is replaced by the short, flexible, linker GGSGSGEGSGSGG (SEQ ID NO: 1) (ait1Dc3 and ait1Dc4, respectively). Both mutant proteins fold and are trafficked to the vacuolar membrane, as judged by GFP-AitDC3 and GFP-AitDC4 localization (FIG. 8s). However, in line with the C3 loop model, only aitDc3 cells had a defect in TORC1 inhibition during leucine starvation (FIG. 8b).

To further test our model, a strain with three hydrophobic residues in the putative Gtr1/2 binding sequence of the C3 loop mutated to alanine (I217A, I226A, V230A, Ait1c3v1; FIG. 8a) was created. As predicted, ait1c3v1 cells had a significant defect in TORC1 repression during leucine starvation (FIG. 8b). Moreover, the mutations in the ait1c3v1 strain did not disrupt TORC1 localization during log phase growth (FIG. 8c), indicating that while the tip of C3 loop acts to regulate TORC1 activity via Gtr1/2, it is the body of Ait1 that acts to hold TORC1 in position around the vacuolar membrane.

### Example 6 Materials and Methods

### Strain Construction

All strains used in this study were generated in haploid (W303) S. cerevisiae, using standard methods^{69,70}, and are listed in Table S3.

### Crosslinking and Immunopurification

Yeast carrying Kog1-FLAG, and separately Kog1-HA, were inoculated into 5 mL of synthetic complete medium containing 2% glucose (SD medium), and grown overnight at 30° C. in a 20 mL tube, rotating at 40 rpm. The cells were then: (1) Diluted to an OD₆₀₀ of 0.1 in 250 mL of fresh SD medium, and grown shaking at 200 rpm and 30° C. in a 1 L flask, until they reached an OD₆₀₀ of 0.6; (2) Captured by filtration, washed with 2×100 mL of the appropriate stress or starvation medium, and transferred into 200 mL of synthetic medium lacking all nitrogen (-N), all glucose (-Glu), SD medium containing 0.4 M KCl or 1 mM H₂O₂, or SD medium at 42° C.; (3) Grown again for the indicated period of time (FIG. 1), shaking at 200 rpm and 30° C. (or 42° C. for heat stress) in a 1 L flask; (4) Harvested by filtration, and rinsed into 2 mL screw-cap tubes using a small volume of immunoprecipitation lysis buffer (IPLB; 20 mM HEPES, pH 7.5, 150 mM potassium acetate, 2 mM magnesium acetate, 1 mM EGTA, and 0.6 M sorbitol)⁵⁵; and (5) centrifuged for 30 s at 8000 rpm, the supernatant discarded, and the pellet flash frozen, and stored at −80° C.

To lyse the cells, the frozen pellets were resuspended in approximately 600 μL of IPLB buffer containing protease and phosphatase inhibitors (Roche, Indianapolis, IN; 04693159001 and 04906845001; IPLB⁺⁺), and 1 mL of glass microbeads, and the slurries subjected to 6×1 min of vigorous shaking in a Mini-Beadbeater-24 (BioSpec) at 4° C. The tubes were then punctured using a 23 gauge needle and the lysates eluted into 1.5 mL tubes by centrifugation at 3,000 rpm at 4° C., for 5 minutes. The lysates were then homogenized by gentle vortexing, decanted into a fresh 1.5 ml tube, and treated with 0.25 μM of dithiobis (succinimidyl propionate) (DSP) at 4° C. for 30 min (with gentle rotation). At this point crosslinking was then quenched by adding 70 μL of 1 M Tris-HCl, pH 7.5, to each tube and holding the extracts on ice for 30 minutes. Finally, 1% digitonin was added to each tube, and the extracts incubated at 4° C. for 1 hour (with gentle rotation), before they were clarified by centrifugation at 12,000 rpm at 4° C., for 10 minutes, and the supernatant transferred into a fresh tube.

To purify Kog1 and any crosslinked interactors, 50 μL of μMACS anti-FLAG beads (Miltenyi Biotech, 130-101-591) was added to each clarified extract, and the tubes rotated at 4° C. for 1.5 hours. The μMACS columns were then prepared by washing them with 200 μL of the lysis buffer supplied with the purification kit, followed by 200 μL of IPLB⁺⁺ containing 1% digitonin, before the bead/extract mix was loaded into each column (on a magnet) and allowed to flow through by gravity. The beads were then washed in three steps: (1) four times with 200 μL of IPLB++ containing 0.1% digitonin, (2) two times with 400 μL of IPLB++ containing no digitonin, and (3) once with 200 μL of 20 mM Tris-HCl, pH 7.5. Kog1 and any crosslinked proteins were then eluted by incubating each column with 20 μL of the elution buffer supplied with the kit (heated to 95° C.), for 5 minutes, and then adding of 2×40 μL of the same elution buffer containing 50 mM DTT (also at 95° C.). The pooled eluate from each column was then loaded into a single lane on a 10% SDS polyacrylamide gel and allowed to migrate until it completely entered the gel. The gels were then stained with colloidal blue, destained, and the lane excised for analysis by mass spectrometry.

Identical procedures were used to identify Pib2 and Ait1 interactors, except that in these experiments the IP was done using GFP-Pib2 or Ait1-GFP and anti-GFP beads (Miltenyi Biotech, 130-101-125).

### Protein Identification by Mass Spectrometry

Gel slices were washed with water, 50% acetonitrile/50% water, acetonitrile, ammonium bicarbonate (100 mM), and then 50% acetonitrile/50% ammonium bicarbonate (100 mM). The liquid was then removed from each sample, and the gel slices dried in a speed vac. The gel slices were then: (i) Reduced with dithiothreitol (10 mM in 100 mM ammonium bicarbonate) at 56° C. for 45 min, and the solution removed and discarded. (ii) Alkylated with iodoacetamide (55 mM in 100 mM ammonium bicarbonate) in the dark at ambient temperature for 30 min. (iii) Washed with ammonium bicarbonate (100 mM) for 10 min on a shaker, an equal volume of acetonitrile added, and washed for an additional 10 min on a shaker, and then dried in a speed vac for 45 min. (iv) Cooled on ice and a treated with a cold solution of 12.5 ng/uL trypsin (Promega, Madison, WI) in ammonium bicarbonate (100 mM). After 45 min, the trypsin solution was removed and discarded, and an equal amount of ammonium bicarbonate (50 mM) was added, and the sample incubated overnight at 37° C. with mixing. The samples were then spun down in a microfuge and the supernatants collected. Peptides were further extracted from the gel slices by adding 0.1% trifluoroacetic acid (TFA; enough to cover the gel slices) and mixed at ambient temperature for 30 min. An equal amount of acetonitrile was then added, and the samples were mixed for an additional 30 min. The samples were then spun on a microfuge and the supernatants pooled and concentrated in a speed vac. Finally, all samples were desalted using ZipTip C₁₈ (Millipore, Billerica, MA) and eluted with 70% acetonitrile/0.1% TFA, and concentrated in a speed vac.

For analysis, the peptide samples were brought up in 2% acetonitrile and 0.1% formic acid (10 μL) and analyzed (8 μL) by LC/ESI MS/MS with a Thermo Scientific Easyl000 nLC (Thermo Scientific, Waltham, MA) coupled to a hybrid Orbitrap Fusion (Thermo Scientific, Waltham, MA) mass spectrometer. In-line de-salting was accomplished using a reversed-phase trap column (100 μm×20 mm) packed with Magic C₁₈AQ (5-μm 200 Å resin; Michrom Bioresources, Auburn, CA) followed by peptide separations on a reversed-phase column (75 μm×250 mm) packed with Magic C₁₈AQ (5-μm 100 Å resin; Michrom Bioresources, Auburn, CA) directly mounted on the electrospray ion source. A 90-minute gradient from 2% to 35% acetonitrile in 0.1% formic acid at a flow rate of 300 nL/minute was used for chromatographic separations. A spray voltage of 2000 V was applied to the electrospray tip and the Orbitrap Fusion instrument was operated in the data-dependent mode, MS survey scans were in the Orbitrap (AGC target value 500,000, resolution 120,000, and injection time 50 ms) with a 3 sec cycle time and MS/MS spectra acquisition were detected in the linear ion trap (AGC target value of 10,000 and injection time 35 ms) using HCD activation with a normalized collision energy of 27%. Selected ions were dynamically excluded for 45 seconds after a repeat count of 1.

Data analysis was performed using Proteome Discoverer 2.2 (Thermo Scientific, San Jose, CA). The data were searched against an SGD yeast database that included common contaminants. Searches were performed with settings for the proteolytic enzyme trypsin. Maximum missed cleavages was set to 2. The precursor ion tolerance was set to 10 ppm and the fragment ion tolerance was set to 0.6 Da. Variable modifications included oxidation on methionine (+15.995 Da) and carbamidomethyl (57.021). Sequest HT was used for database searching. All search results were run through Percolator for scoring.

### Fluorescence Microscopy

TORC1-body formation was measured as described previously^{47,48}. Briefly, stains carrying Kog1-YFP were patched from their glycerol stocks onto fresh YEPD plates and grown overnight at 30° C. The patches were then used to inoculate 5 mL of SD medium, and the tubes grown at 30° C. in a 20 mL tube, rotating at 40 rpm, until they reached an OD₆₀₀ of 0.1. These starter cultures were then used to inoculate 20 mL of SD medium in a 150 ml Erlenmeyer flask (to an OD₆₀₀ below 0.01) and grown at 30° C. and shaking at 200 rpm, until they reached an and OD₆₀₀ between 0.5 and 0.7. 300 μL of each culture was then pipetted into one chamber in an 8-well micro-slide (Ibidi, 80826) that had been pretreated with concanavalin A. The chambers were then washed three times with SD-nitrogen, and images acquired using a Nikon Eclipse Ti-E microscope equipped with a 100× objective, a Photometrics Prime 95B camera, and λ_{EX} 510/25 and λ_{EM} 540/21 filters, every 10 min for an hour. Each image consisted of a z-stack of sixteen 200 ms images, spaced 0.4 μm apart, to ensure that the bodies in all planes were detected, and was compressed into a maximum projection stack in ImageJ for analysis.

Imaging of GFP-Ait1, Gtr1-YFP, GFP-Pib2 was done in an identical manner except that GFP images were acquired λEX 470 and λEM 515/30 filters.

### Rps6 Phosphorylation Assays

Cultures were grown in conical flasks shaking at 200 rpm and 30° C. until mid-log phase (OD₆₀₀ between 0.55 and 0.6). At this point, a 47 mL sample was collected, mixed with 3 mL 100% trichloroacetic acid (TCA), and held on ice for at least 30 min (and up to 6 hrs). The remaining culture was then collected by filtration, and transferred to SD-N, SD-aa, or SD-leu medium after two washes with 100 ml of the same medium, and further samples collected in TCA, as described above. The samples were then centrifuged at 4000 rpm for 5 min at 4° C., washed twice with 4° C. water, twice with acetone, and disrupted by sonication at 15% amplitude for 5 s before centrifugation at 8000 rpm for 30 s. The cell pellets were then dried in a speedvac for 10 min at room temperature, and frozen until required at −80° C.

Protein extraction was performed by bead beating (6×1 min, full speed) in urea buffer (6 M urea, 50 mM tris-HCl pH 7.5, 5 mM EDTA, 1 mM PMSF, 5 mM NaF, 5 mM NaN₃, 5 mM NaH₂PO₄, 5 mM p-nitrophenylphosphate, 5 mM β-glycerophosphate, 1% SDS) supplemented with complete protease and phosphatase inhibitor tablets (Roche, Indianapolis, IN; 04693159001 and 04906845001). The lysate was then harvested by centrifugation for 5 min at 3000 rpm, resuspended into a homogenous slurry, and heated at 65° C. for 10 min. The soluble proteins were then separated from insoluble cell debris by centrifugation at 12,000 rpm for 10 min, and the lysate stored at −80° C. until required.

For protein phosphorylation analysis, the protein extracts were run on a 12% acrylamide gel and transferred to a nitrocellulose membrane. Western blotting was then carried out using anti-pRPS6 antibody (Cell Signaling, 4858) at a 1/2500 dilution, and anti-PGK1 antibody (Invitrogen, 459250) at a 1/10,000 dilution, and anti-mouse and anti-rabbit secondaries, labeled with a IRDye 700CW and IRDye 800CW (LiCor), both at a 1/10000 dilution, and the blots scanned using a LiCor Odyssey Scanner (LiCor, Lincon, NE). Band intensities were quantified using the LiCor Image Studio Software.

### Sch9 Bandshift Experiments

Sch9 bandshift measurements were performed as described previously^{71,72}, and using the same procedure listed above for the Rps6 Western, except that lysates were subjected to cleavage by 2-nitro-5-thiocyanatobenzoic acid (NTCB) for 12-16 hrs at room temperature (1 mM NTCB and 100 mM CHES, pH 10.5) prior to analysis, and the Western was done using an anti-HA (12CA5) antibody.

### LITERATURE CITED

All references cited in this disclosure, including but not limited to those listed below, are hereby incorporated by reference into this disclosure.
1 Gonzalez, A. & Hall, M. N. Nutrient sensing and TOR signaling in yeast and mammals. EMBO J 36, 397-408, doi:10.15252/embj.201696010 (2017).
2 Loewith, R. & Hall, M. N. Target of rapamycin (TOR) in nutrient signaling and growth control. Genetics 189, 1177-1201, doi:10.1534/genetics.111.133363 (2011).
3 Liu, G. Y. & Sabatini, D. M. mTOR at the nexus of nutrition, growth, ageing and disease. Nat Rev Mol Cell Biol 21, 183-203, doi:10.1038/s41580-019-0199-y (2020).
4 Huber, A. et al. Characterization of the rapamycin-sensitive phosphoproteome reveals that Sch9 is a central coordinator of protein synthesis. Genes Dev 23, 1929-1943, doi:10.1101/gad.532109 (2009).
5 Robitaille, A. M. et al. Quantitative phosphoproteomics reveal mTORC1 activates de novo pyrimidine synthesis. Science 339, 1320-1323, doi:10.1126/science.1228771 (2013).
6 Hsu, P. P. et al. The mTOR-regulated phosphoproteome reveals a mechanism of mTORC1-mediated inhibition of growth factor signaling. Science 332, 1317-1322, doi:10.1126/science.1199498 (2011).
7 Peterson, T. R. et al. mTOR complex 1 regulates lipin 1 localization to control the SREBP pathway. Cell 146, 408-420, doi:10.1016/j.cell.2011.06.034 (2011).
8 Ben-Sahra, I. & Manning, B. D. mTORC1 signaling and the metabolic control of cell growth. Curr Opin Cell Biol 45, 72-82, doi:10.1016/j.ceb.2017.02.012 (2017).
9 Ben-Sahra, I., Hoxhaj, G., Ricoult, S. J. H., Asara, J. M. & Manning, B. D. mTORC1 induces purine synthesis through control of the mitochondrial tetrahydrofolate cycle. Science 351, 728-733, doi:10.1126/science.aad0489 (2016).
10 Kim, J., Kundu, M., Viollet, B. & Guan, K. L. AMPK and mTOR regulate autophagy through direct phosphorylation of Ulk1. Nat Cell Biol 13, 132-141, doi:10.1038/ncb2152 (2011).
11 Kamada, Y. et al. Tor-mediated induction of autophagy via an Apg1 protein kinase complex. J Cell Biol 150, 1507-1513, doi:10.1083/jcb.150.6.1507 (2000).
12 Duvel, K. et al. Activation of a metabolic gene regulatory network downstream of mTOR complex 1. Mol Cell 39, 171-183, doi:10.1016/j.molcel.2010.06.022 (2010).
13 Barbet, N. C. et al. TOR controls translation initiation and early G1 progression in yeast. Mol Biol Cell 7, 25-42 (1996).
14 Manning, B. D., Tee, A. R., Logsdon, M. N., Blenis, J. & Cantley, L. C. Identification of the tuberous sclerosis complex-2 tumor suppressor gene product tuberin as a target of the phosphoinositide 3-kinase/akt pathway. Mol Cell 10, 151-162, doi:10.1016/s1097-2765(02)00568-3 (2002).
15 Inoki, K., Li, Y., Zhu, T., Wu, J. & Guan, K. L. TSC2 is phosphorylated and inhibited by Akt and suppresses mTOR signalling. Nat Cell Biol 4, 648-657, doi:10.1038/ncb839 (2002).
16 Menon, S. et al. Spatial control of the TSC complex integrates insulin and nutrient regulation of mTORC1 at the lysosome. Cell 156, 771-785, doi:10.1016/j.cell.2013.11.049 (2014).
17 Dibble, C. C. & Manning, B. D. Signal integration by mTORC1 coordinates nutrient input with biosynthetic output. Nat Cell Biol 15, 555-564, doi:10.1038/ncb2763 (2013).
18 Yang, H. et al. Mechanisms of mTORC1 activation by RHEB and inhibition by PRAS40. Nature 552, 368-373, doi:10.1038/nature25023 (2017).
19 Inoki, K., Zhu, T. & Guan, K. L. TSC2 mediates cellular energy response to control cell growth and survival. Cell 115, 577-590, doi:10.1016/s0092-8674(03)00929-2 (2003).
20 Inoki, K., Li, Y., Xu, T. & Guan, K. L. Rheb GTPase is a direct target of TSC2 GAP activity and regulates mTOR signaling. Genes Dev 17, 1829-1834, doi:10.1101/gad.1110003 (2003).
21 Gwinn, D. M. et al. AMPK phosphorylation of raptor mediates a metabolic checkpoint. Mol Cell 30, 214-226, doi:10.1016/j.molcel.2008.03.003 (2008).
22 Sancak, Y. et al. Ragulator-Rag complex targets mTORC1 to the lysosomal surface and is necessary for its activation by amino acids. Cell 141, 290-303, doi:10.1016/j.cell.2010.02.024 (2010).
23 Sancak, Y. et al. The Rag GTPases bind raptor and mediate amino acid signaling to mTORC1. Science 320, 1496-1501, doi:10.1126/science. 1157535 (2008).
24 Kim, E., Goraksha-Hicks, P., Li, L., Neufeld, T. P. & Guan, K. L. Regulation of TORC1 by Rag GTPases in nutrient response. Nat Cell Biol 10, 935-945, doi: 10.1038/ncb1753 (2008).
25 Bar-Peled, L., Schweitzer, L. D., Zoncu, R. & Sabatini, D. M. Ragulator is a GEF for the rag GTPases that signal amino acid levels to mTORC1. Cell 150, 1196-1208, doi:10.1016/j.cell.2012.07.032 (2012).
26 Efeyan, A. et al. Regulation of mTORC1 by the Rag GTPases is necessary for neonatal autophagy and survival. Nature 493, 679-683, doi:10.1038/nature11745 (2013).
27 Rogala, K. B. et al. Structural basis for the docking of mTORC1 on the lysosomal surface. Science 366, 468-475, doi:10.1126/science.aay0166 (2019).
28 Bar-Peled, L. et al. A Tumor suppressor complex with GAP activity for the Rag GTPases that signal amino acid sufficiency to mTORC1. Science 340, 1100-1106, doi:10.1126/science.1232044 (2013).
29 Wolfson, R. L. et al. Sestrin2 is a leucine sensor for the mTORC1 pathway. Science 351, 43-48, doi:10.1126/science.aab2674 (2016).
30 Saxton, R. A. et al. Structural basis for leucine sensing by the Sestrin2-mTORC1 pathway. Science 351, 53-58, doi:10.1126/science.aad2087 (2016).
31 Saxton, R. A., Chantranupong, L., Knockenhauer, K. E., Schwartz, T. U. & Sabatini, D. M. Mechanism of arginine sensing by CASTOR1 upstream of mTORC1. Nature 536, 229-233, doi:10.1038/nature19079 (2016).
32 Chantranupong, L. et al. The CASTOR Proteins Are Arginine Sensors for the mTORC1 Pathway. Cell 165, 153-164, doi:10.1016/j.cell.2016.02.035 (2016).
33 Gu, X. et al. SAMTOR is an S-adenosylmethionine sensor for the mTORC1 pathway. Science 358, 813-818, doi:10.1126/science.aao3265 (2017).
34 Wang, S. et al. Metabolism. Lysosomal amino acid transporter SLC38A9 signals arginine sufficiency to mTORC1. Science 347, 188-194, doi:10.1126/science.1257132 (2015).
35 Castellano, B. M. et al. Lysosomal cholesterol activates mTORC1 via an SLC38A9-Niemann-Pick C1 signaling complex. Science 355, 1306-1311, doi:10.1126/science.aag1417 (2017).
36 Tatebe, H. & Shiozaki, K. Evolutionary Conservation of the Components in the TOR Signaling Pathways. Biomolecules 7, doi:10.3390/biom7040077 (2017).
37 Binda, M. et al. The Vam6 GEF controls TORC1 by activating the EGO complex. Mol Cell 35, 563-573, doi:10.1016/j.molcel.2009.06.033 (2009).
38 Dubouloz, F., Deloche, O., Wanke, V., Cameroni, E. & De Virgilio, C. The TOR and EGO protein complexes orchestrate microautophagy in yeast. Mol Cell 19, 15-26, doi:10.1016/j.molcel.2005.05.020 (2005).
39 Zhang, T. et al. Structural insights into the EGO-TC-mediated membrane tethering of the TORC1-regulatory Rag GTPases. Sci Adv 5, eaax8164, doi: 10.1126/sciadv.aax8164 (2019).
40 Powis, K. et al. Crystal structure of the Ego1-Ego2-Ego3 complex and its role in promoting Rag GTPase-dependent TORC1 signaling. Cell Res 25, 1043-1059, doi:10.1038/cr.2015.86 (2015).
41 Zhang, T., Peli-Gulli, M. P., Yang, H., De Virgilio, C. & Ding, J. Ego3 functions as a homodimer to mediate the interaction between Gtr1-Gtr2 and Ego1 in the ego complex to activate TORC1. Structure 20, 2151-2160, doi:10.1016/j.str.2012.09.019 (2012).
42 Panchaud, N., Peli-Gulli, M. P. & De Virgilio, C. Amino acid deprivation inhibits TORC1 through a GTPase-activating protein complex for the Rag family GTPase Gtr1. Sci Signal 6, ra42, doi:10.1126/scisignal.2004112 (2013).
43 Neklesa, T. K. & Davis, R. W. A genome-wide screen for regulators of TORC1 in response to amino acid starvation reveals a conserved Npr2/3 complex. PLoS Genet 5, e1000515, doi:10.1371/journal.pgen.1000515 (2009).
44 Chen, J., Sutter, B. M., Shi, L. & Tu, B. P. GATOR1 regulates nitrogenic cataplerotic reactions of the mitochondrial TCA cycle. Nat Chem Biol 13, 1179-1186, doi:10.1038/nchembio.2478 (2017).
45 Laxman, S., Sutter, B. M., Shi, L. & Tu, B. P. Npr2 inhibits TORC1 to prevent inappropriate utilization of glutamine for biosynthesis of nitrogen-containing metabolites. Sci Signal 7, ra120, doi:10.1126/scisignal.2005948 (2014).
46 Algret, R. et al. Molecular architecture and function of the SEA complex, a modulator of the TORC1 pathway. Mol Cell Proteomics 13, 2855-2870, doi:10.1074/mcp.M114.039388 (2014).
47 Hughes Hallett, J. E., Luo, X. & Capaldi, A. P. Snf1/AMPK promotes the formation of Kog1/Raptor-bodies to increase the activation threshold of TORC1 in budding yeast. Elife 4, doi:10.7554/eLife.09181 (2015).
48 Sullivan, A., Wallace, R. L., Wellington, R., Luo, X. & Capaldi, A. P. Multilayered regulation of TORC1-body formation in budding yeast. Mol Biol Cell 30, 400-410, doi:10.1091/mbc.E18-05-0297 (2019).
49 Varlakhanova, N. V., Mihalevic, M. J., Bernstein, K. A. & Ford, M. G. J. Pib2 and the EGO complex are both required for activation of TORC1. J Cell Sci 130, 3878-3890, doi:10.1242/jcs.207910 (2017).
50 Kim, A. & Cunningham, K. W. A LAPF/phafin1-like protein regulates TORC1 and lysosomal membrane permeabilization in response to endoplasmic reticulum membrane stress. Mol Biol Cell 26, 4631-4645, doi:10.1091/mbc.E15-08-0581 (2015).
51 Ukai, H. et al. Gtr/Ego-independent TORC1 activation is achieved through a glutamine-sensitive interaction with Pib2 on the vacuolar membrane. PLoS Genet 14, e1007334,doi:10.1371/journal.pgen.1007334 (2018).
52 Tanigawa, M. & Maeda, T. An In Vitro TORC1 Kinase Assay That Recapitulates the Gtr-Independent Glutamine-Responsive TORC1 Activation Mechanism on Yeast Vacuoles. Mol Cell Biol 37, doi:10.1128/MCB.00075-17 (2017).
53 Michel, A. H. et al. Functional mapping of yeast genomes by saturated transposition. Elife 6, doi:10.7554/eLife.23570 (2017).
54 Shen, X. X. et al. Tempo and Mode of Genome Evolution in the Budding Yeast Subphylum. Cell 175, 1533-1545 e1520, doi:10.1016/j.cell.2018.10.023 (2018).
55 Murley, A. et al. Sterol transporters at membrane contact sites regulate TORC1 and TORC2 signaling. J Cell Biol 216, 2679-2689, doi:10.1083/jcb.201610032 (2017).
56 Liu, Z., Sekito, T., Spirek, M., Thornton, J. & Butow, R. A. Retrograde signaling is regulated by the dynamic interaction between Rtg2p and Mks1p. Mol Cell 12, 401-411, doi:10.1016/s1097-2765(03)00285-5 (2003).
57 Breitkreutz, A. et al. A global protein kinase and phosphatase interaction network in yeast. Science 328, 1043-1046, doi:10.1126/science.1176495 (2010).
58 Alliance of Genome Resources, C. Alliance of Genome Resources Portal: unified model organism research platform. Nucleic Acids Res 48, D650-D658, doi:10.1093/nar/gkz813 (2020).
59 UniProt, C. UniProt: a worldwide hub of protein knowledge. Nucleic Acids Res 47, D506-D515, doi:10.1093/nar/gky1049 (2019).
60 Wilson, Z. N., Scott, A. L., Dowell, R. D. & Odorizzi, G. PI(3,5)P2 controls vacuole potassium transport to support cellular osmoregulation. Mol Biol Cell 29, 1718-1731, doi:10.1091/mbc.E18-01-0015 (2018).
61 Chen, X. et al. Whi2 is a conserved negative regulator of TORC1 in response to low amino acids. PLoS Genet 14, e1007592, doi:10.1371/journal.pgen.1007592 (2018).
62 Yerlikaya, S. et al. TORC1 and TORC2 work together to regulate ribosomal protein S6 phosphorylation in Saccharomyces cerevisiae. Mol Biol Cell 27, 397-409,doi:10.1091/mbc.E15-08-0594 (2016).
63 Bonfils, G. et al. Leucyl-tRNA synthetase controls TORC1 via the EGO complex. Mol Cell 46, 105-110, doi:10.1016/j.molcel.2012.02.009 (2012).
64 Wyant, G. A. et al. mTORC1 Activator SLC38A9 Is Required to Efflux Essential Amino Acids from Lysosomes and Use Protein as a Nutrient. Cell 171, 642-654 e612,doi:10.1016/j.cell.2017.09.046 (2017).
65 Lei, H. T., Ma, J., Sanchez Martinez, S. & Gonen, T. Crystal structure of arginine-bound lysosomal transporter SLC38A9 in the cytosol-open state. Nat Struct Mol Biol 25, 522-527,doi:10.1038/s41594-018-0072-2 (2018).
66 Fromm, S. A., Lawrence, R. E. & Hurley, J. H. Structural mechanism for amino acid-dependent Rag GTPase nucleotide state switching by SLC38A9. Nat Struct Mol Biol 27, 1017-1023, doi:10.1038/s41594-020-0490-9 (2020).
67 Shen, K., Choe, A. & Sabatini, D. M. Intersubunit Crosstalk in the Rag GTPase Heterodimer Enables mTORC1 to Respond Rapidly to Amino Acid Availability. Mol Cell 68, 821, doi:10.1016/j.molcel.2017.10.031 (2017).
68 Fukuda, T. et al. Tripartite suppression of fission yeast TORC1 signaling by the GATOR1-Sea3 complex, the TSC complex, and Gon2 kinase. Elife 10, doi:10.7554/eLife.60969 (2021).
69 Storici, F. & Resnick, M. A. The delitto perfetto approach to in vivo site-directed mutagenesis and chromosome rearrangements with synthetic oligonucleotides in yeast. Methods Enzymol 409, 329-345, doi:10.1016/S0076-6879(05)09019-1 (2006).
70 Storici, F., Lewis, L. K. & Resnick, M. A. In vivo site-directed mutagenesis using oligonucleotides. Nat Biotechnol 19, 773-776, doi:10.1038/90837 (2001).
71 Urban, J. et al. Sch9 is a major target of TORC1 in Saccharomyces cerevisiae. Mol Cell 26, 663-674, doi:10.1016/j.molcel.2007.04.020 (2007).
72 Hughes Hallett, J. E., Luo, X. & Capaldi, A. P. State transitions in the TORC1 signaling pathway and information processing in Saccharomyces cerevisiae. Genetics 198, 773-786, doi:10.1534/genetics.114.168369 (2014).
73 Omasits, U., Ahrens, C. H., Muller, S. & Wollscheid, B. Protter: interactive protein feature visualization and integration with experimental proteomic data. Bioinformatics 30, 884-886, doi:10.1093/bioinformatics/btt607 (2014).

We claim:

1. A composition for treating a fungal infection, comprising an engineered Ait1 protein or fragment thereof, said engineered Ait1 protein or fragment thereof binding to TORC1 with an altered affinity than wildtype Ait1 protein from same strain.

2. The composition of claim 1, wherein the engineered Ait1 protein or fragment thereof binds to TORC1 with a higher affinity than wildtype Ait1 protein from same strain.

3. The composition of claim 1, wherein the engineered Ait1 protein or fragment thereof comprises one or more mutations.

4. A method for treating a fungal infection in a subject in need thereof, comprising administering to the subject the composition of claim 1.

5. The method of claim 4, wherein the subject has contracted fungal infection caused by Candida glabrata.

6. The method of claim 5, wherein the engineered Ait1 protein or fragment thereof slows down growth of the Candida glabrata in the subject.

7. A composition for treating a fungal infection, comprising an agent that binds to Ait1 protein or fragment thereof.

8. The composition of claim 7, wherein said agent binds to an Ait1 protein or fragment thereof endogenous to a pathogenic Candida glabrata strain.

9. A method for treating a fungal infection in a subject, comprising administering to the subject the composition of claim 7.

10. The method of claim 9, wherein the subject has contracted fungal infection caused by Candida glabrata.

11. The method of claim 10, wherein the Candida glabrata is resistant to drug treatment.

12. The method of claim 10, wherein the agent slows down growth of the Candida glabrata in the subject.

13. A method of modulating growth of a yeast cell having an endogenous Ait1 gene, said method comprising
a) adding an agent to a culture comprising said yeast cell, said agent binding to the endogenous Ait1 gene in the yeast cell, and
b) allowing said agent to enter said yeast cell and modulate growth rate of said yeast cell.

14. The method of claim 13, wherein the agent slows down growth rate of said yeast cell.

15. The method of claim 13, wherein the agent binds to the endogenous Ait1 gene.

16. The method of claim 13, wherein the yeast cell is an engineered yeast cell that that is engineered to produce a chemical or a non-native protein.

17. The method of claim 16, wherein the agent reduces the growth rate of the yeast cell and increases production of the chemical or the non-native protein.

18. A method of modulating growth of a yeast cell having an endogenous Ait1 gene, said method comprising introducing one or more mutations into the endogenous Ait1 gene to generate an engineered yeast cell, said one or more mutations causing the engineered yeast cells to grow faster or slower than a wildtype yeast cell with the same genetic background other than the one or more mutations in the Ait1 gene.

19. A method of screening for a candidate compound effective in treating fungal infection, comprising
a) contacting said plurality of compounds with Ait1 protein or fragment thereof; and
b) selecting the candidate compound that binds to the Ait1 protein or fragment thereof or alter retention of TORC1 by Ait1.

20. The method of claim 19, wherein readout in step (b) is altered localization of TORC1.


SEQUENCE LISTING (9 sequences)

SEQ ID NO: 1 - protein, 13 aa, synthetic construct
GGSGSGEGSGSGG

SEQ ID NO: 2 - protein, 547 aa, synthetic construct
MVRLNHAASYFMPIFCSTRPHIVILSALFSISLFSLFYASSELLLHQYDDPLMFKPNSQDYFRTFLLGLFSPFLYYFLKTFLFNINQRFLILNLIVDFPINDVFMLLILIGLAYPQVQDHEGGTIKHKECSWHIIPRQAYIFGISWALGEFTICIIGNLFNYQEIADPNINSGFTHQESANTYCNNNDMSHNDDCGCSTEYRPNVVDRSDITLSKCIEVRNDSSSISNNVYSSEYHPIKPLRSSSSTYGSIRQQPHENKKQLHVPDNSQDDTIIMMNPIDNSLKLTTLDTGDLSFPIDEEQPILKKSFGYTWAVPNENTQNTTKSFTPIKRFIAFSTAYQLVTGLLLMILVVGSNIMLTIGESLILSMYFVYVRGHEGLFTPVVNYFGSRTISNFILCVIIPFISLNFLINTSIYLRRELDDWFNNSQGEFEDDDENTISKRVATNQEYQHPLSANYISMDSPDVINSSPGHFGMNSGQLLGNTTLYYGSLNGDDDDMTNDSALLRFCKKLVKNWRALARNDSFVLGVMVSWSLLVFVTGILSTVYI

SEQ ID NO: 3 - protein, 115 aa, synthetic construct
MANMNSDSRHLGTSEVDHERDPGPMNIQFEPSDLRSKRPFCIEPTNIVNVNHVIQRVSDHASAMNKRIHYYSRLTTPADKALIAPDHVVPAPEECYVVSPLGSAYKLQSYTEGYG

SEQ ID NO: 4 - protein, 176 aa, synthetic construct
NYQEIADPNINSGFTHQESANTYCNNNDMSHNDDCGCSTEYRPNVVDRSDITLSKCIEVRNDSSSISNNVYSSEYHPIKPLRSSSSTYGSIRQQPHENKKQLHVPDNSQDDTIIMMNPIDNSLKLTTLDTGDLSFPIPILKKSFGYTWAVPNENTQNTTKSFTPIKRFIAFSTAYQ

SEQ ID NO: 5 - protein, 42 aa, synthetic construct
DITLSKCIEVRNDSSSISNNVYSSEYHPIKPLRSSSSTYGSI

SEQ ID NO: 6 - protein, 42 aa, synthetic construct
DITASKAAAVANDSSSISNNVYSSEYHPIKPLRSSSSTYGSI

SEQ ID NO: 7 - protein, 42 aa, synthetic construct
DATASKAAAVANDSSSASNNVYSSEYHPIKPLRSSSSTYGSI

SEQ ID NO: 8 - protein, 42 aa, synthetic construct
DITLSKCIEVRNDSSSISNNVYSSEYHPIKPLRAAAAAAAAA

SEQ ID NO: 9 - DNA, 1644 bp, Saccharomyces cerevisiae
  source 1..1644: mol_type = other DNA
atggtccgattaaatcatgcagcatcatattttatgcctattttctgctccacaaggcctcacatagtgatactttctgcgctgtttagtatttcactgttttcactattctatgcgtcgtccgaattactgctacaccagtatgatgatccattgatgttcaaaccaaattcgcaggactatttcaggacctttcttttggggcttttttcaccatttctttattatttcctgaagacctttctattcaatataaatcaaaggtttttaatattgaacctgatagtggattttcctatcaacgacgtctttatgcttttgatactgattggtttagcgtaccctcaggtgcaagatcacgaaggcggtacgatcaagcataaggaatgttcttggcacatcattccaagacaagcctatatctttggtatatcatgggccctgggtgaattcaccatatgcataatagggaatttgtttaactaccaagaaatcgccgatccaaacattaatagtggcttcacacaccaggaaagcgccaacacttactgtaacaataatgatatgagccataacgatgactgcggttgtagtacagaatatcgccccaacgtcgtagacagaagtgatattacgttgtccaaatgtattgaagtaagaaatgactcatcttccatatcgaataacgtatactcgtctgagtaccatccaatcaaacctctacgttcatcgtcatcaacttatggcagcatacgacagcaacctcatgaaaataagaagcagctgcatgtaccagataattcgcaggacgatacaattatcatgatgaaccccatcgacaattcattaaagttgacaactctagatacgggtgatttgagtttccccatagacgaagaacagcccattttgaagaaatcttttggttatacatgggcagttcctaatgaaaatacacaaaatactactaaaagcttcacaccgataaagagatttattgcatttagcacggcataccagttggtcaccggtttattgttaatgatattggtagttggtagtaatatcatgttgacaattggggaatcattgattctgtcgatgtatttcgtctatgttcgcggccatgaaggattatttactcccgtggtgaactacttcggctcaagaaccatttcaaatttcatcttatgcgtaataattcctttcatatctctaaacttcctcatcaacacttcgatatatttaagaagagagctagacgattggtttaataattcacagggggagtttgaggacgacgacgaaaatactatcagtaagagagtagctacaaatcaagagtaccaacatccactgagtgctaattacatttcgatggatagtcctgatgtaataaatagcagcccgggtcacttcgggatgaattcaggccagttgttaggtaatacgacgttatattatggtagtttaaatggggatgatgatgatatgaccaacgactccgctttactaagattctgcaagaagctagtcaaaaattggagggctttggcaagaaatgattcttttgtacttggagtaatggtttcttggagtcttcttgtttttgtaacgggaatactttcaacagtttatatatag